(19) United States
(12) Patent Application Publication
(10) Pub. No.: US 2013/0178908 A1
(43) Pub. Date: Jul. 11, 2013

(54) ELECTROPHYSIOLOGICAL ENDOCARDIOLOGY TOOL

(71) Applicant: Endoheart AG, Winterthur (CH)

(72) Inventor: Christoph Hans Huber, Bern (CH)

(73) Assignee: ENDOHEART AG, Winterthur (CH)

(21) Appl. No.: 13/737,151

(22) Filed: Jan. 9, 2013

Related U.S. Application Data
(60) Provisional application No. 61/631,639, filed on Jan. 9, 2012.

(51) Int. Cl. A61N 1/362; A61N 1/05

(52) U.S. Cl. CPC A61N 1/3621; A61N 1/056
USPC 607/14; 607/122

(57) ABSTRACT

Apparatus and methods for pacing the heart. The apparatus may include, and the methods may involve, an elongated member having: a delivery lumen that is configured to traverse the heart wall, the lumen having a proximal opening for receiving the instrument and a distal opening for deploying the instrument; and an electrically conductive member that is configured to deliver to the heart wall a current that modifies a contraction frequency. The apparatus may include an access opening closure device that has: a distal end that is configured to be disposed interior the heart and contact endocardial tissue adjacent the access opening; and a proximal end that is configured to be disposed exterior the heart and contact heart tissue adjacent the access opening; and an electrode that is configured to discharge electrical energy into the heart wall to change the frequency. The apparatus may include an injectable needle pacing electrode.

### CROSS-REFERENCE TO OTHER APPLICATIONS

This application is a nonprovisional of U.S. Application No. 61/631,639 which was filed on Jan. 9, 2012, and is hereby incorporated by reference herein in its entirety.

### BACKGROUND

Rapid pacing of the heart is often used during therapeutic activities in the heart. The rapid pacing may reduce blood pressure and flow in the heart. This may reduce stress on heart anatomy when therapeutic instruments or prostheses are present in the heart and may interfere with normal blood flow through the heart. For example, when instruments or prostheses are “upstream” from a valve and partially or wholly obstruct flow through the valve, destructive stress can be transmitted to the valve and adjacent tissue.

Rapid pacing of the heart often requires applying current from at least two electrodes. Often, a first electrode is placed in contact with the heart and a second electrode is place in contact with the patient's skin. Open surgery provides easy access to heart tissue for deployment of an electrode. Smaller access incisions that are used in percutaneous procedures, however, reduce access to the heart tissue and make it more difficult to apply an electrode to the heart surface or to retain the electrode on the heart surface during the procedure.

In some procedures, the heart surface electrode may be replaced by a temporary venous pacing lead that is deployed in a chamber of the heart, for example, a right atrium or a right ventricle. The pacing lead may be deployed via a neck, arm or leg vein.

Temporary venous pacing may require intervention that is not required by placement of the heart surface electrode.

It would be desirable to provide apparatus and methods for rapid pacing of the heart in connection with percutaneous procedures.

### BRIEF DESCRIPTION OF THE DRAWINGS

The objects and advantages of the invention will be apparent upon consideration of the following detailed description, taken in conjunction with the accompanying drawings, in which like reference characters refer to like parts throughout, and in which:

FIG. 1 shows apparatus in accordance with the principles of the invention along with illustrative anatomy, in cross-section, in connection with which the invention may be practiced;

FIG. 2 is a cross-sectional view, oriented like that along lines 2-2 (shown in FIG. 1), of other apparatus in accordance with the principles of the invention along with anatomy.

FIG. 3 shows other apparatus in accordance with the principles of the invention along with anatomy;

FIG. 4 shows the apparatus and anatomy of FIG. 3 when the apparatus is in a position different from that shown in FIG. 3;

FIG. 5 shows the apparatus and anatomy of FIG. 3 when the apparatus is in a different position and a different configuration from the position and configuration shown in FIG. 3;

FIG. 6 shows still other apparatus in accordance with the principles of the invention along with anatomy;

FIG. 7 shows the apparatus of FIG. 6 when the apparatus in a different position from the position shown in FIG. 6;

FIG. 8 shows yet other apparatus in accordance with the principles of the invention along with anatomy;

FIG. 9 shows the apparatus of FIG. 8 along with yet other apparatus and anatomy.

FIG. 10 shows the apparatus of FIG. 8 along with yet other apparatus and anatomy;

FIG. 11 shows the apparatus of FIG. 8 along with yet other apparatus in accordance with the principles of the invention, and yet other apparatus, and anatomy;

FIG. 12 shows, in partial cross-section, oriented like that along lines 12-12 (shown in FIG. 1), of other apparatus in accordance with the principles of the invention;

FIG. 13 shows a cross-section taken along lines 13-13 (shown in FIG. 12) of the apparatus shown in FIG. 12;

FIG. 14 shows a cross-section, oriented like that along lines 13-13 (shown in FIG. 12), of other apparatus in accordance with the principles of the invention;

FIG. 15 shows, in partial cross-section, oriented like that along lines 15-15 (shown in FIG. 1), of other apparatus in accordance with the principles of the invention;

FIG. 16 shows a cross-section taken along lines 16-16 (shown in FIG. 15) of the apparatus shown in FIG. 15;

FIG. 17 shows yet other apparatus in accordance with the principles of the invention along with anatomy;

FIG. 18 shows a portion, indicated in FIG. 17 as “18,” of the apparatus shown in FIG. 17;

FIG. 19 shows, in partial cross-section, yet other apparatus in accordance with the principles of the invention along with anatomy;

FIG. 20 shows a portion, indicated in FIG. 20 as “19,” of the apparatus shown in FIG. 19;

FIG. 21 shows yet other apparatus in accordance with the principles of the invention along with anatomy;

FIG. 22 shows a cross-section taken along lines 22-22 (shown in FIG. 21) of the apparatus shown in FIG. 21 along with anatomy;

FIG. 23 shows yet other apparatus in accordance with the principles of the invention along with anatomy;

FIG. 24 shows a cross-section taken along lines 24-24 (shown in FIG. 23) of the apparatus shown in FIG. 23 along with anatomy;

FIG. 25 shows a cross-section taken along lines 25-25 (shown in FIG. 24) of the apparatus shown in FIG. 23 along with anatomy;

FIG. 26 shows, in partial cross-section, yet other apparatus in accordance with the principles of the invention, that corresponds to a portion of the apparatus shown in FIG. 23 and indicated in FIG. 24 as “26;”

FIG. 27 shows the apparatus of FIG. 8 along with yet other apparatus and anatomy;

FIG. 28 shows the apparatus of FIG. 8 along with yet other apparatus and anatomy;

FIG. 29 shows the apparatus of FIG. 8 along with yet other apparatus and anatomy;

FIG. 30 shows yet other apparatus that may be used in accordance with the principles of the invention;

FIG. 31 shows the apparatus of FIG. 8 along with yet other apparatus and anatomy;

FIG. 32 shows the apparatus of FIG. 8 along with yet other apparatus and anatomy;

FIG. 33 shows the apparatus of FIG. 8 along with yet other apparatus and anatomy;

FIG. 34 shows yet other apparatus that may be used in accordance with the principles of the invention;

FIG. 35 shows yet other apparatus that may be used in accordance with the principles of the invention;

FIG. 36 shows, in partial cross-section, yet other apparatus in accordance with the principles of the invention;

FIG. 37 shows a cross-section taken along lines 37-37 (shown in FIG. 36) of the apparatus shown in FIG. 36; and

FIG. 38 shows a cross-section, oriented like that along lines 37-37 (shown in FIG. 36), of other apparatus in accordance with the principles of the invention.

### DETAILED DESCRIPTION

Apparatus and method for delivering an instrument through an access opening in an exterior heart wall are provided. The apparatus may include an elongated member. The elongated member may include a delivery lumen that is configured to traverse the heart wall. The lumen may have a proximal opening for receiving the instrument and a distal opening for deploying the instrument. The apparatus may include an outer surface that is configured to maintain the access opening. The apparatus may include an electrically conductive member. The electrically conductive member may be supported by the elongated member. The electrically conductive member may be configured to deliver to the heart wall a current that modifies a contraction frequency of the heart. The apparatus may include one, two, three, four, 10 or more, or any suitable number of electrically conductive members. The electrically conductive member may be an electrode.

The electrically conductive member may be used to provide current to the heart in conjunction with another electrically conductive member that is placed elsewhere in the heart, on the heart, or on the patient's skin and also provides pacing current to the patient's tissue.

The elongated member may include any suitable biocompatible material such as polymer, stainless steel, nickel titanium alloy or any other suitable material.

The apparatus may include, for each electrically conductive member, a current supply lead. The current supply lead may receive one or more cardiac pacing signals from a cardiac pacing signal generator. A connector may be provided for placing the current supply lead in electrical communication with the cardiac pacing signal generator. The cardiac pacing signal generator may include any suitable pacing device.

In some procedures, more than one of the apparatus may be used together. For example, a first instrument having electrically conductive members for transferring pulses to the heart and a second instrument having electrically conductive members for transferring pulses to the heart may be coaxially arranged, the first inside the second. The first instrument may be extend from the distal end of the second instrument and be advanced into the myocardium to perform a first procedure. During the first procedure, pulses may be transferred to the heart from the first instrument.

After the first procedure, the second instrument may be advance along the first instrument into the myocardium. When the second instrument advances into the myocardium, pulses may be transferred to the heart from the second instrument. A current switch may be provided to transfer electrical energy from the first instrument to the second instrument. The current switch may analyze an electrical characteristic of one or both of the first and second instruments to detect the succession of the second instrument in the access opening. The current switch may deactivate the first instrument and activate the second instrument upon or about the time of the succession. The electrical characteristic may include a continuity. The electrical characteristic may include an impedance.

Table 1 lists illustrative instruments that may be delivered using the apparatus.

TABLE 1
Illustrative instruments that may be delivered using the apparatus.
Illustrative instrument

Ablation catheter
Access closure device
Aneurysm sealing device
Aortic endostent
Catheter
Coronary stent
Decalcification tool
Defibrillator
Delivery system
Embolic protection device
Embolism preventer
Endoprosthesis
Heart assist device
Heart valve
Heat sink
Heat source
Introducer
Microwave emitter
Needle
Occluder device
Pacemaker
Plug tool
Radio frequency radiation emitter
Valve
Valve reconstruction tool (e.g., clip)
Valve removal tool
Wire

Table 2 lists illustrative procedures that may be carried out using the apparatus.

TABLE 2
Illustrative procedures that may be carried out using the apparatus.
Illustrative procedure

Aneurysm repair
Cardiac defibrillation
Cardiac pacing
Coronary dilatation
Coronary stenting
Decalcification of cardiac structures
Endocardiac arrhytHₘia mapping
Heart and hemodynamic support
Heart valve reconstruction
Heart valve repair
Hemodynamic diagnostic assessments
Hemodynamic recording
Removal of hypertrophic obstructions (obstructions distal to a heart valve)
Repair dissected blood vessel
Repair ruptured blood vessel
Replacement of dissected blood vessel
Replacement of ruptured blood vessel
Resynchronization therapy
Stemcell therapy
Transcatheter valve implantation
Transcatheter valve insertion
Transcatheter valve replacement
Treatment of subvalvular stenosis (an obstruction proximal to a heart
valve)
Ventricular arrhytHₘia repair

The electrically conductive member may be configured to provide to the heart wall a series of pulses. The pulses may be quantified by pacing parameters. The pacing parameters may include voltage, current, energy, duration, pulse frequency, maxima and minima thereof, and any other suitable pacing parameters.

Table 3 shows illustrative ranges of some pacing parameters.

TABLE 3
Illustrative ranges of pulse current.

| | | |
|---|---|---|
| Current | Pulse duration | Pulse frequency |

| From | To | From | To | From | To |
|---|---|---|---|---|---|
| about | about | about | about | about | about |
| (mA) | (mA) | (ms) | (ms) | (Hz) | (Hz) |
| 0.01 | 0.05 | 0.01 | 0.05 | 1 | 3 |
| 0.05 | 0.1 | 0.05 | 0.1 | 3 | 10 |
| 0.1 | 1 | 0.1 | 1 | 10 | 30 |
| 1 | 3 | 1 | 3 | 30 | 60 |
| 3 | 10 | 3 | 10 | 60 | 100 |
| 10 | 20 | 10 | 20 | 100 | 130 |
| 20 | 30 | 20 | 40 | 130 | 160 |
| 30 | 40 | 40 | 60 | 160 | 200 |
| 40 | 50 | 60 | 80 | 200 | 230 |
| 50 | 60 | 80 | 100 | 230 | 260 |
| 60 | 100 | 100 | 200 | 260 | 300 |

Each pulse may carry from about 0.1 to about 40 milliamp (“mA”). Each pulse may have a duration that is in the range of about 0.1 to about 100 millisecond (“ms”). The pulses may be delivered with a frequency of about 10 to about 300 pulses per second.

The electrically conductive member may include copper, silver, gold, platinum, polymer or any other suitable conductive material. The electrically conductive member may include conductive wire, tape, foil, sheet, rod, bar, tube, shot or any other suitable form.

The electrically conductive member may be configured to be in direct contact with the heart wall. The electrically conductive member may be configured to be in indirect contact with the heart wall. The electronically conductive member may rest upon the outer surface. The electronically conductive member may be partially or wholly recessed into the elongated member. The electronically conductive member may be partially or wholly recessed relative to the outer surface into the elongated member.

The outer surface may be configured to slide through the access opening. When the outer surface slides through the access opening, the distal opening may displace from a first location to a second location. The first location may be in the heart, in a first chamber of the heart or in the vasculature. The second location may be in the heart, in a first chamber of the heart, in a second chamber of the heart or in the vasculature. The first chamber may be an aorta or a ventricle. The second chamber may be an aorta or a ventricle. The vasculature may include an artery. The vasculature may include a vein. The artery may be a pulmonary artery, a carotid artery, a coronary artery or any other suitable artery. The vein may a vena cava, a pulmonary vein, a coronary vein or any other suitable vein.

The electrically conductive member may be configured to slide through the access opening while the electrically conductive delivers the current to the heart wall. The electrically conductive member may be configured to be in direct contact with the heart wall while the electrically conductive member slides through the access opening. Contact between the electrically conductive member and the heart wall may be largely independent of positioning of the elongated member in the heart wall.

The apparatus may include an antenna that is supported by the elongated member and is configured to sense a native cardiac electric field in a chamber on the interior side of the heart wall. The antenna may communicate a corresponding native cardiac signal to a receiver exterior the heart wall. The receiver may be part of an electrocardiograph device. The antenna may communicate the cardiac signal via a transmission line. The antenna may communicate the cardiac signal wirelessly.

The apparatus may include a pressure sensor. The pressure sensor may be supported by the elongated member. The pressure sensor may be supported by the electrically conductive member. The pressure sensor may be configured to sense a pressure in the chamber. The pressure sensor may be configured to sense a pressure in the heart wall. The pressure sensor may be configured to transmit a corresponding pressure signal to a receiver exterior the heart wall. The antenna may communicate the pressure signal via a transmission line. The antenna may communicate the pressure signal wirelessly.

The apparatus may include a chemical sensor. The chemical sensor may be supported by the elongated member. The chemical sensor may be supported by the electrically conductive member. The chemical sensor may be configured to measure chemical values such as, for example, pH, lactate, cardiac enzymes, electrolytes. The chemical sensor may be configured to transmit a corresponding signal to a receiver exterior the heart wall. The chemical sensor may transmit the chemical signal via a transmission line. The chemical sensor may transmit the chemical signal wirelessly.

The chemical sensor may be calibrated to sense a chemical species. The species may be present in a chamber interior the heart wall. The species may be present at a myocardial tissue surface that is exposed in a heart wall access opening and transmit a corresponding chemical signal to a receiver exterior the heart wall.

The chemical sensor may detect the chemical value based on conductivity of the heart wall. The chemical sensor may detect the chemical value based on capacitance of the heart wall. The chemical sensor may detect the chemical value based on an electrical potential of the heart wall. The chemical sensor may include a porous layer. The chemical sensor may detect the chemical value based on conductivity of the porous layer. The chemical sensor may detect the chemical value based on capacitance of the porous layer. The chemical sensor may detect the chemical value based on an electrical potential of the porous layer.

The apparatus may include a processor that is configured to change a pacing parameter, for example, a frequency of the current based on the native cardiac signal. The processor may be configured to change the pacing parameter based on one or more pressure signal. The processor may be configured to change the pacing parameter based on one or more chemical signal.

The outer surface may apply pressure to the heart wall. The pressure may be sufficient to prevent blood transmission between the outer surface and the heart wall. The outer surface may apply contact pressure to the heart wall. The pressure may substantially prevent blood leakage between the outer surface and the heart wall by resisting pressure from the heart muscle upon the outer surface. The blood transmission may be prevented in the absence of a sealing element, proximate the access opening, on an interior side of the heart wall.

The elongated member may be configured to be maintained in a position, relative to the access opening, without an anchor that fixes the elongated member to the heart wall. When the elongated member is maintained in the position, it may be maintained such that it counters pressure of blood inside the heart.

The elongated member may include a proximal portion that is configured to be grasped by any suitable stabilization structure. The elongated member may have a longitudinal position, relative to the access opening, that is maintained primarily by the structure. The elongated member may have a longitudinal position, relative to the access opening, that is maintained exclusively by the structure. The structure may be a human hand. The structure may be a robotic manipulator.

The electrically conductive member may be configured to be released from the elongated member and inserted in the heart wall. The electrically conductive member may be inserted into the endocardium. The electrically conductive member may be inserted into the myocardium. The electrically conductive member may be inserted into the pericardium. The electrically conductive member may be fixed onto the endocardium. The electrically conductive member may be fixed onto the myocardium. The electrically conductive member may be fixed onto the pericardium.

The electrically conductive member may be anchored in the heart wall. The electrically conductive member may be anchored by a barb, a coil or any other suitable anchor. The electrically conductive member may be a wire. The wire may have a distal end that is driven into the heart wall. The electrically conductive member may be configured to be released from the elongated member and placed on the heart wall. The electrically conductive member may be left in place in the heart wall after removal of the elongated member from the access opening. The electrically conductive member may later be removed from the heart.

The elongated member may include a deployment lumen that is configured to direct the electrically conductive member into the heart wall. The elongated member may include an annular wall about the delivery lumen. The deployment lumen may run longitudinally within the annular wall. The deployment lumen may terminate at an orifice in the outer surface.

The elongated member may not include a deployment lumen. For example, when the elongated member is solid, as in a wire or a non-cannulated rod, the elongated member may not include a deployment lumen.

The wire may be part of a delivery system for delivering a prosthetic valve into the heart. When the wire is part of the delivery system, the prosthetic valve may be crimped onto a distal tip of the wire. The wire, with the valve, may be inserted into the access opening. The outer surface of the wire may be in contact with the heart wall. The contact may be direct contact. The wire, with the valve, may be inserted through the access opening within a sheath. The sheath may maintain the valve in an unexpanded state until the valve is in position for deployment. The sheath may be withdrawn proximally to allow a distal end of the valve to expand and engage heart anatomy. Further withdrawal of the sheath may allow a proximal end of the valve to expand and engage heart anatomy.

The wire, with the valve, may be inserted through an introducer. The introducer may have an outer surface that is in contact with the heart wall. The contact may be direct contact.

The apparatus may include one, two, three, four, 10 or more, or any suitable number of deployment lumens. Each of the deployment lumens may correspond to one or more electrically conductive members.

The elongated member may include a distal surface that is configured to abut pericardial tissue adjacent the access opening. The deployment lumen may terminate at an orifice in the distal surface.

The apparatus may include a control link. The control link may be configured to displace the electrically conductive member along the deployment lumen and into the heart wall. The control link may be configured to detach from the electrically conductive member when the electrically conductive member is lodged in the heart wall.

The control link may urge the electrically conductive member by contacting the electrically conductive member at a location on the electrically conductive member that is distal a portion of the electrically conductive member that initially engages the heart wall. The control link may urge the electrically conductive member by contacting the electrically conductive member at a location on the electrically conductive member that is proximal a portion of the electrically conductive member that initially engages the heart wall.

The apparatus may include a conductor that is attached to the electrically conductive member and runs proximally from the electrically conductive member through the deployment lumen.

The apparatus may include a second deployment lumen. The second deployment lumen may be configured to direct the electrically conductive member into the heart wall. The second deployment lumen may run longitudinally, with respect to the elongated member, within an annular wall about the delivery lumen. The second deployment lumen may terminate at an orifice in the outer surface.

The apparatus may include a second electrically conductive member that is supported by the elongated member. The second electrically conductive member may be configured to deliver to the heart wall a current that modifies the contraction frequency.

The electrically conductive member may be embedded in the outer surface. The electrically conductive member may be substantially flush with the outer surface. The electrically conductive member may extend away from the outer surface. The electrically conductive member may extend radially, relative to a longitudinal axis of the elongated member, away from the outer surface.

The apparatus may include a blood retention diaphragm having an instrument orifice. The instrument orifice may be expandable. The diaphragm may be disposed in the delivery lumen. The diaphragm may be positioned in the access opening.

The elongated member includes a needle.

The outer surface may have, in a distal region, a distal diameter; and, in a proximal region, a proximal diameter that is greater than the distal diameter.

The elongated member may include a catheter. The catheter may have an inside diameter in the range of about 0.1 to about 3 millimeter (“mm”); and an outside diameter in the range of about 1 to about 4 mm. The inside diameter may have any suitable length. The outside diameter may have any suitable length.

The catheter may be part of a delivery system for delivering a prosthetic valve into the heart. When the catheter is part of the delivery system, the prosthetic valve may be crimped onto a distal tip of the catheter. The catheter, with the valve, may be inserted into the access opening. The outer surface of the catheter may be in contact with the heart wall. The contact may be direct contact. The catheter, with the valve, may be inserted through the access opening within a sheath. The sheath may maintain the valve in an unexpanded state until the valve is in position for deployment. The sheath may be withdrawn proximally to allow a distal end of the valve to expand and engage heart anatomy. Further withdrawal of the sheath may allow a proximal end of the valve to expand and engage heart anatomy.

The catheter, with the valve, may be inserted through an introducer. The introducer may have an outer surface that is in contact with the heart wall. The contact may be direct contact.

The catheter may be any suitable size, including about 33Fr to about 24Fr and any suitable sizes below, within or above that range.

The elongated member may include an introducer. The introducer may have an inside diameter in the range of about 0.1 to about 18 mm; and an outside diameter in the range of about 5 to about 20 mm. The inside diameter may have any suitable length. The outside diameter may have any suitable length.

A portion of the elongated member may be in a contracted state. The contracted state may be a mechanical equilibrium state. When the portion of the elongated member is in the contracted state, a corresponding portion of the electrically conductive member may be disposed at a first radial distance from a longitudinal central axis of the delivery lumen. The portion of the elongated member may be changed to an expanded state. When the portion of the elongated member is in the expanded state, the corresponding portion of the electrically conductive member may be disposed at a second radial distance from the axis. The second radial distance may be greater than the first radial distance.

The portion of the electronically conductive member may be a first portion of the electronically conductive member. When the portion of the elongated member is in the expanded state, a second portion of the electrically conductive member, which may be disposed longitudinally away from the first portion, may be disposed at the first radial distance from the axis.

The elongated member may include material that is configured to deform radially in response to displacement through the delivery lumen of an instrument having an outer diameter that is greater than an inner diameter of the lumen.

The material may be configured to elastically relax after passage of the instrument. Relaxation of the material may return the first portion of the electrically conductive member substantially to the first radial distance.

The material is configured so that the inner diameter remains deformed substantially commensurate with the outer diameter of the instrument after passage of the instrument.

The instrument that displaces the elongated member may be a blunt rod. The instrument that displaces the elongated member may be a therapeutic device. The instrument that displaces the elongated member may be a prosthetic heart valve or any other suitable therapeutic device such as one or more of the devices described or shown herein.

Apparatus and methods for plugging the access opening are provided. The apparatus may include a body. The body may include a distal end. The distal end may be configured to be disposed interior the heart. The distal end may be configured to contact endocardial tissue adjacent the access opening. The apparatus may include a proximal end. The proximal end may be configured to be disposed exterior the heart. The proximal end may be configured to contact pericardial tissue adjacent the access opening. The apparatus may include an electrode. The electrode may be supported by the body. The electrode may be configured to discharge electrical energy into the heart wall to change a frequency of contraction of the heart. The apparatus may include any suitable number of electrodes.

The plug may include, for each electrode, a current supply lead. The current supply lead may receive one or more cardiac pacing signals from a cardiac pacing signal generator. A connector may be provided for placing the current supply lead in electrical communication with the cardiac pacing signal generator. The cardiac pacing signal generator may include any suitable pacing device.

The body may include a stem that extends between the distal end and the proximal end. The body may include a shaft that extends between the distal end and the proximal end. The shaft may have one or more features in common with the stem. The stem may have a first diameter. The distal end may have a second diameter. The second diameter may be greater than the first diameter. The proximal end may have a third diameter. The third diameter may be greater than the first diameter.

The electrode may discharge from the distal end. The electrode may discharge from the proximal end. The electrode may discharge from the stem. The electrode may discharge from the shaft.

The body may include a second electrode. The second electrode may be configured to discharge electrical energy into the heart wall to change the frequency of contraction of the heart.

The body may include a sensor for sensing a native cardiac electric field.

The body may include, in the distal end, a sensor for sensing a blood pressure inside the heart.

The body may include a sensor for measuring chemical values inside the heart. The sensor may be disposed in the distal end. The sensor may be disposed on the stem. The sensor may be disposed on the shaft. The chemical sensor may be supported by the elongated member. The chemical sensor may be supported by the electrically conductive member. The chemical sensor may be configured to measure chemical values such as, for example, pH, lactate, cardiac enzymes, electrolytes.

The chemical sensor may be calibrated to sense a chemical species. The species may be present in a chamber interior the heart wall. The species may be present at a myocardial tissue surface that is exposed in a heart wall access opening.

The chemical sensor may detect the chemical value based on conductivity of the heart wall. The chemical sensor may detect the chemical value based on capacitance of the heart wall. The chemical sensor may detect the chemical value based on an electrical potential of the heart wall. The chemical sensor may include a porous layer. The chemical sensor may detect the chemical value based on conductivity of the porous layer. The chemical sensor may detect the chemical value based on capacitance of the porous layer. The chemical sensor may detect the chemical value based on an electrical potential of the porous layer.

The sensor may transmit a chemical value signal based on the chemical value to a processor outside the heart. The sensor may transmit the chemical value sensor by wire. The sensor may transmit the chemical value sensor wirelessly.

The body may include an electrical energy storage source such as a battery. The body may include a pacing signal generator. The battery may supply electrical current to the electrodes. The signal generator may control the current so that the current is provided in a therapeutic form.

The battery may be separate from the body. The battery may be separately implantable in the patient and in wired electrical communication with the body.

The battery may be inductively recharged from a source exterior the patient.

The apparatus may include a processor that is configured to change a pacing parameter, for example, a frequency of the current based on the native cardiac signal. The processor may be configured to change the pacing parameter based on one or more pressure signal. The processor may be configured to change the pacing parameter based on one or more chemical signal.

The methods may include inserting an elongated member through an access opening in the heart. The methods may include transferring electrical energy from the elongated member to the heart to change a frequency of contractions of the heart. The methods may include delivering an instrument through the elongated member.

The transferring may include discharging the electrical energy across a contact interface between the elongated member and the heart.

The method may include sensing an electric field of the heart at the elongated member. The method may include changing the contraction frequency based on the electric field.

The method may include sensing a blood pressure in the heart at the elongated member. The method may include changing the frequency based on the pressure.

The method may include sensing a chemical value in the heart at the elongated member. The method may include changing the contraction frequency based on the chemical value.

The method may include preventing blood loss through the access opening by maintaining the elongated member in the access opening.

The delivering may include delivering a guide wire.

The delivering may include delivering a catheter.

The delivering may include delivering an introducer. The delivering may include delivering a introducer. The introducer may include a hemostatic valve.

The delivering may include delivering an ultrasound receiver.

The delivering may include delivering an embolic protection device.

The delivering may include delivering a valve resection device. The resection device may be used to resect a heart valve. The resection device may be used to treat a hypertrophic obstruction. The resection device may be used to treat a subvalvular obstruction.

The delivering may include delivering a prosthetic valve. The valve may be a stent valve. The stent valve may be self-expanding. The stent valve may be balloon-expandable.

The method may include deploying from the elongated member an access opening plug.

The method may include transferring electrical energy from the plug to the heart to change the frequency of contractions of the contractions.

The method may include displaying a live image of the elongated member as positioned inside the access opening. The method may include displaying, adjacent the live image, a native cardiac electric field trace. The method may include displaying, adjacent the live image, an internal cardiac blood pressure trace.

Apparatus for pacing a heart with an injectable needle are provided. The apparatus may include a sharp end that is configured to pierce tissue; a base opposite the sharp end, the base being configured to receive an impulse to drive the sharp end into the tissue; an elongated member bearing an electrode, the elongated member extending from the sharp end to the base; and an electrically conducting lead that is connected to the electrode and is configured to receive current for delivery to the heart through the electrode when the electrode is embedded in the heart.

The electrode may be elongated along a longitudinal direction of the elongated member.

When the electrode is a first electrode and the electrically conducting lead is a first electrically conducting lead, the apparatus may include a second electrode that is borne by the elongated member; and a second electrically conducting lead that is connected to the electrode and is configured to receive current for delivery to the heart through the second electrode when the second electrode is embedded in the heart.

The base may be configured to receive the impulse from a firing peg, a spring, a pressurized gas, or any other suitable impulse source.

The base may include a magnet. When the base includes a magnet, the base may be configured to receive the impulse from a magnetic field. The magnetic field may be provided by a coil.

Apparatus and methods in accordance with the invention will now be described in connection with the Figures. The features are illustrated in the context of selected embodiments. It will be understood that features shown in connection with one of the embodiments may be practiced in accordance with the principles of the invention along with features shown in connection with others of the embodiments.

Apparatus and methods described herein are illustrative. Apparatus and methods of the invention may involve some or all of the features of the illustrative apparatus and/or some or all of the steps of the illustrative methods. The steps of the methods may be performed in an order other than the order shown and described herein. Some embodiments may omit steps shown and described in connection with the illustrative methods. Some embodiments may include steps that are not shown and described in connection with the illustrative methods.

The apparatus and methods of the invention will be described in connection with embodiments and features of illustrative heart treatment devices and associated hardware and instrumentation. The device and associated hardware and instruments will be described now with reference to the FIGS. It is to be understood that other embodiments may be utilized and structural, functional and procedural modifications may be made without departing from the scope and spirit of the present invention.

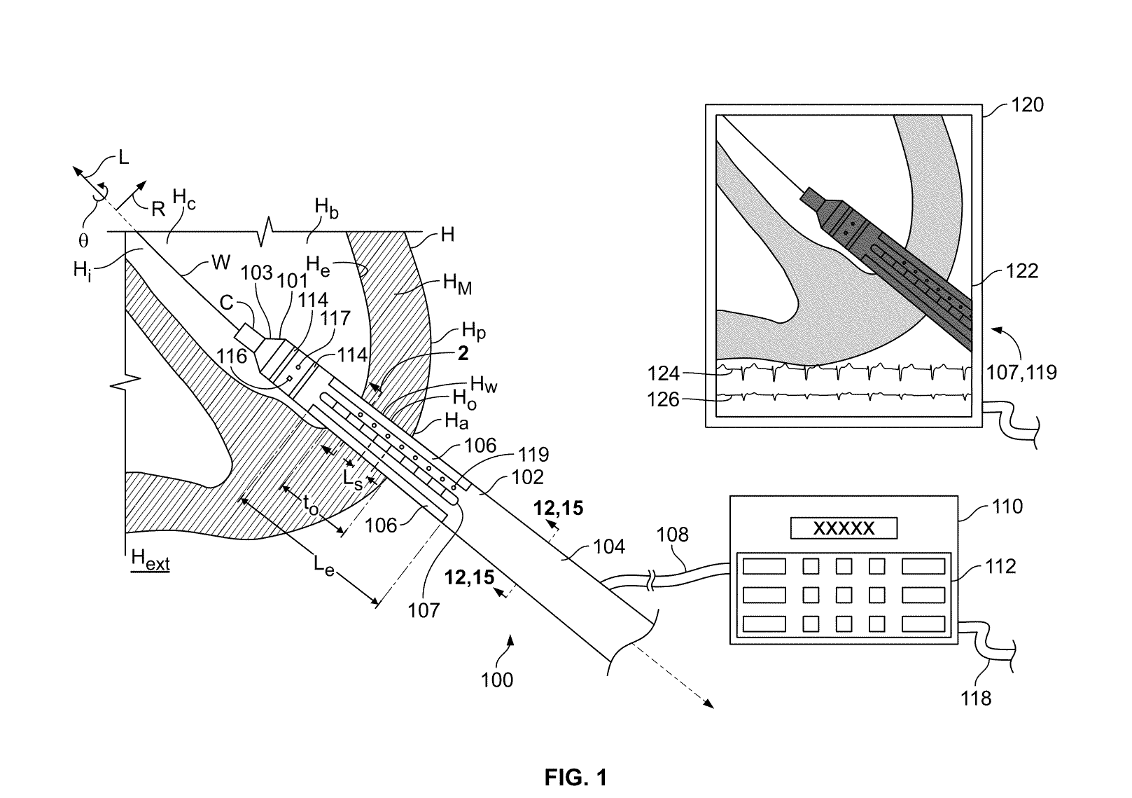

FIG. 1 schematically shows illustrative instrument 100 for performing a procedure on a heart such as heart H. The procedure may be performed on anatomy in or about heart H. The procedure may be performed on anatomy in or about a chamber of heart H such as chamber H_{c}. Chamber H_{c} may be a left ventricle, a right ventricle, a left atrium or a right atrium. The procedure may be performed on vasculature in or about heart H or on any other structure in or about heart H. Heart H may contract at a frequency.

Heart H may include pericardium Hₚ, myocardium Hₘ and endocardium Hₑ. Heart H may include apex Ha. Heart H may include heart wall H. Heart wall H_{w} may include one or more of pericardium Hₚ, myocardium Hₘ and endocardium Hₑ. Heart wall H_{w} may include a septum between two cardiac atria. Heart wall H_{w} may include a septum between two cardiac ventricles.

Instrument 100 may include elongated member 102. Elongated member 102 may be cannulated. Elongated member 102 may include outer surface 104. Outer surface 104 may resist closure of access opening Hₒ in heart wall H. The pressure of heart wall H_{w} on outer surface 104 may be sufficient to prevent the leakage of blood H_{b} from interior H, of chamber H, between outer surface 104 and heart wall H_{w} to exterior Hₑₓₜ of heart H. The pressure may be sufficient to do so even in the absence of a seal at the contact between outer surface 104 and heart wall H.

Instrument 100 may include one or more electrically conductive members such as representative electrically conductive member 106. One or more of electrically conductive members 106 may be supported by elongated member 102. One or more of electrically conductive members 106 may be supported by outer surface 104. One or more of electrically conductive members 106 may be used to provide electrical pulses to heart wall H_{w} to change the contraction frequency. One or more of electrically conductive members 106 may be placed in direct contact with heart wall H_{w} to provide the electrical pulses. The energy may be supplied via a cable such as 108 from source 110. The energy may be supplied wirelessly from source 110. Source 110 may be programmable via control panel 112. Source 110 may be incorporated into instrument 100. Source 110 may be or include a pacing device.

One or more additional electrically conductive members may have a distal end that is placed in electrical communication with epidermal tissue on the body in which the heart is disposed. The one or more additional electrically conductive members may have a proximal end that is in electrical communication with control panel 112.

Instrument 100 may be displaced along longitudinal axis L. Instrument 100 may be rotated in circumferential direction θ. Instrument 100 may be radially tapered, along axis L, with respect to radial direction R. Instrument 100 may deliver the pulses while instrument 100 is moving longitudinally. Instrument 100 may deliver the pulses while instrument 100 is rotating.

One or more of electrically conductive members 106 may remain in direct contact with heart wall H_{w} while instrument 100 is moving longitudinally. One or more of electrically conductive members 106 may remain in direct contact with heart wall H_{w} while instrument 100 is rotating. One or more of electrically conductive members 106 may have a length Le. Access opening Hₒ may have a thickness tₒ. Length Le may be greater than thickness tₒ such that instrument 100 may move longitudinally without removing the electrically conductive member from contact with heart wall H.

One or more of electrically conductive members 106 may sense a native cardiac electric field. A signal corresponding to the field may be transmitted to source 110. The signal may be transmitted via cable 108. The signal may be transmitted wirelessly. Instrument 100 may include one or more electrically conductive members 106 that are wired to provide pulses to heart wall H_{w} and one or more electrically conductive members 106 that are wired to transmit a native cardiac electric field signal to an electrocardiograph device.

Instrument 100 may include one or more antennae such as representative antenna 114. Antennae 114 may sense a native cardiac electric field. A signal corresponding to the field may be transmitted to source 110. The signal may be transmitted via cable 108. The signal may be transmitted wirelessly.

Instrument 100 may include one or more pressure sensors such as representative pressure sensor 116. Pressure sensor 116 may sense a pressure of blood H_{b}. A signal corresponding to the pressure may be transmitted to source 110. The signal may be transmitted via cable 108. The signal may be transmitted wirelessly.

Instrument 100 may include one or more pressure sensors such as representative pressure sensor 119. Pressure sensor 119 may have one or more features in common with pressure sensor 116. Pressure sensor 119 may be one of an array of pressure sensors. Pressure sensor 119 may sense the pressure of heart wall H_{w} against outer surface 104 in access opening Hₒ. Each of the pressure sensors in the array may be monitored individually so that a reading may be taken from one or more of the pressure sensors that is in access opening Hₒ. When elongated member 102 is in different longitudinal positions relative to heart wall H_{w}, it may be necessary or desirable to monitor the pressure sensed by a sensor that is inside access opening Hₒ and not in chamber H_{c} or in the exterior Hₑₓₜ of the heart. In this way, the pressure in access opening H_{c} can be monitored while elongated member 102 is in different positions or is in motion.

Instrument 100 may include one or more chemical sensors such as representative chemical sensor 117. A signal corresponding to the concentration of a chemical may be transmitted to source 110. The signal may be transmitted via cable 108. The signal may be transmitted wirelessly.

Instrument 100 may include one or more chemical sensors such as representative chemical sensor 107. Chemical sensor 107 may have one or more features in common with chemical sensor 117. Chemical sensor 107 may be one of an array of chemical sensors. Chemical sensor 107 may sense the presence at an exposed surface of heart wall H_{w} in access opening Hₒ of a chemical compound or species. Each of the chemical sensors in the array may be monitored individually so that a reading may be taken from one or more of the chemical sensors that is in access opening Hₒ. When elongated member 102 is in different longitudinal positions relative to heart wall H_{w}, it may be necessary or desirable to monitor the chemical value sensed by a sensor that is inside access opening Hₒ and not in chamber H_{c} or in the exterior Hₑₓₜ of the heart. In this way, the chemical value in access opening H_{c} can be monitored while elongated member 102 is in different positions or is in motion.

One or more of the pressure sensors and chemical sensors may have a length Ls. Length Ls may be lesser than thickness tₒ such that when instrument 100 moves longitudinally through heart wall H_{w}, one of the pressure sensors or chemical sensors will sense, respectively, a pressure or chemical signal that is representative of heart wall H_{w}. For example, Ls may be in the range of about tₒ to tₒ/2, tₒ/2 to about tₒ/4, about tₒ/4 to tₒ/8 or smaller.

The pulses may be defined by parameters such as energy, duration, frequency, voltage, current and any other suitable parameters. The parameters may be selected manually via control panel 112. Source 110 may include one or more control algoritHₘs for setting one or more parameters of the pulses. The algoritHₘs may be selected via control panel 112. The algoritHₘs may include settings that may be adjusted via control panel 112.

Instrument 100 may be used to deploy another instrument such as catheter C, guidewire W or any other instrument. One or more of the elements of instrument 100 may be embodied in a needle, a catheter, an introducer or any other instrument that may be involved in the procedure.

When instrument 100 is an introducer, it may be used together with obliterator 101. Obliterator 101 may be a cannulated element that nests coaxially inside instrument 101. Obliterator 101 may include taper 103. Taper 103 may be extended distally of instrument 101 to present taper 103 to access hole Hₒ when access hole Hₒ is too small to accept the diameter of instrument 101. Taper 103 may be used to open access hole Hₒ to accept instrument 101. Obliterator 101 may then be withdrawn proximally from instrument 100 to enable instrumentation to pass through the inside diameter of instrument 100.

Source 110 may be communication, for example, via cable 118, or wirelessly, with other clinical signals. For example, source 110 may exchange information with a medical imaging system (not shown). The medical imaging system may provide an image of instrument 100 during the procedure. The image may be a fluoroscopic image or any other suitable image. Source 110 may provide information such as the native cardiac field, the pressure or any other suitable information. The image and the information may be displayed together on display 120. Display 120 may be collocated with instrument 100, for example, in the same operating theater. Display 120 may be located remotely from instrument 100, for example, at a distant location for observation by a distant practitioner. For example, display 120 may display image 122, electric field trace 124, pressure trace 126 or any other suitable trace. The image and the traces may be live and may be combined into an integrated image signal before being displayed by display 120.

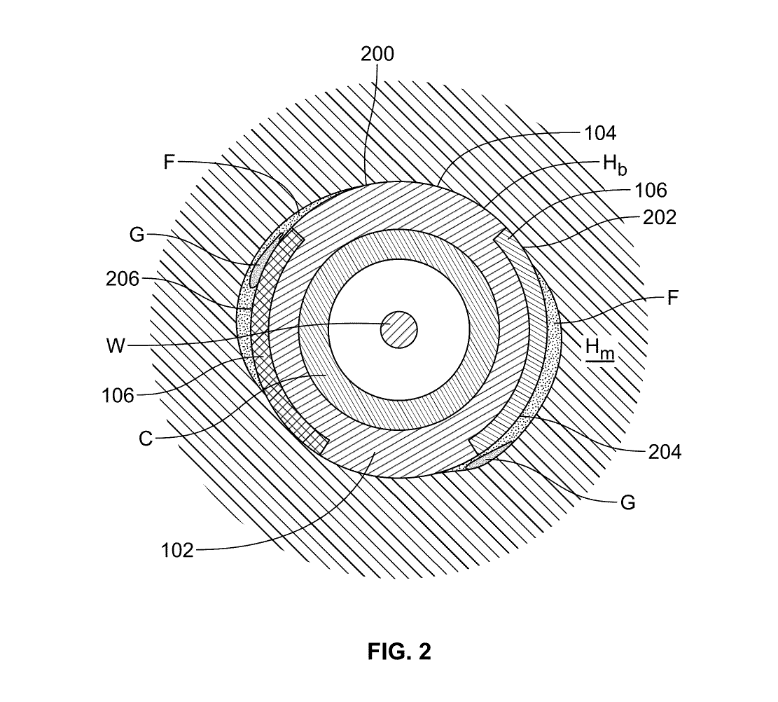

FIG. 2 shows a view taken along lines 2-2 (shown in FIG. 1). FIG. 2 shows elongated member 102 in access opening Hₒ. In region 200, outer surface 104 is in direct contact with myocardium Hₘ. In region 202, one of electrically conductive members 106 is in direct contact with myocardium Hₘ. In region 204, one of electrically conductive members 106 is in indirect contact with myocardium Hₘ. In region 204, fluid F intervenes between the electrically conductive member and myocardium Hₘ. Fluid F may include blood, such as blood H_{b}. Fluid F may include an irrigation fluid, such as water or saline solution. Fluid F may include any other suitable fluid.

In regions 204 and 206, one of electrically conductive members 106 is in indirect contact with myocardium Hₘ. In region 206, fluid F and gas G intervene between the electrically conductive member and myocardium Hₘ. Gas G may include air.

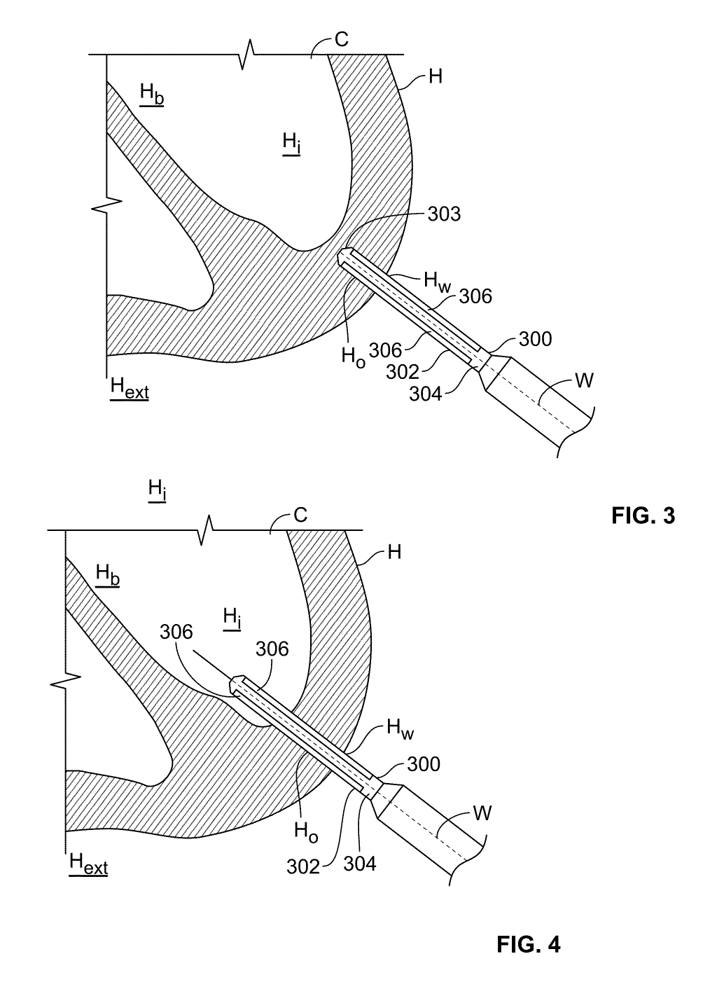

FIGS. 3-10 show illustrative steps of a procedure that may involve apparatus having features such as those illustrated in connection with instrument 100 (shown in FIG. 1).

FIG. 3 schematically shows illustrative access needle 300 penetrating heart wall H_{w} of heart H. Needle 300 may include elongated member 302. Elongated member 302 may include tissue-piercing tip 303. Elongated member 302 may include outer surface 304. Outer surface 304 may resist closure of access opening Hₒ in heart wall H. The pressure of heart wall H_{w} on outer surface 304 may be sufficient to prevent the leakage of blood H_{b} from interior Hᵢ of chamber H_{c} between outer surface 304 and heart wall H_{w} to exterior Hₑₓₜ of heart H. The pressure may be sufficient to do so even in the absence of a seal at the contact between outer surface 304 and heart wall H.

Needle 300 may include one or more electrically conductive members such as representative electrically conductive member 306. One or more of electrically conductive members 306 may be supported by elongated member 302. One or more of electrically conductive members 306 may be supported by outer surface 304. One or more of electrically conductive members 306 may be used to provide electrical pulses to heart wall H_{w} to change the contraction frequency.

Needle 300 may include an delivery lumen (not shown). The delivery lumen may be used to deliver an instrument to interior Hᵢ. The instrument may be any suitable instrument, such as guidewire G.

FIG. 4 shows needle 300 advanced longitudinally through heart wall H_{w}. Electrically conductive members 306 remain in contact with heart wall H_{w}.

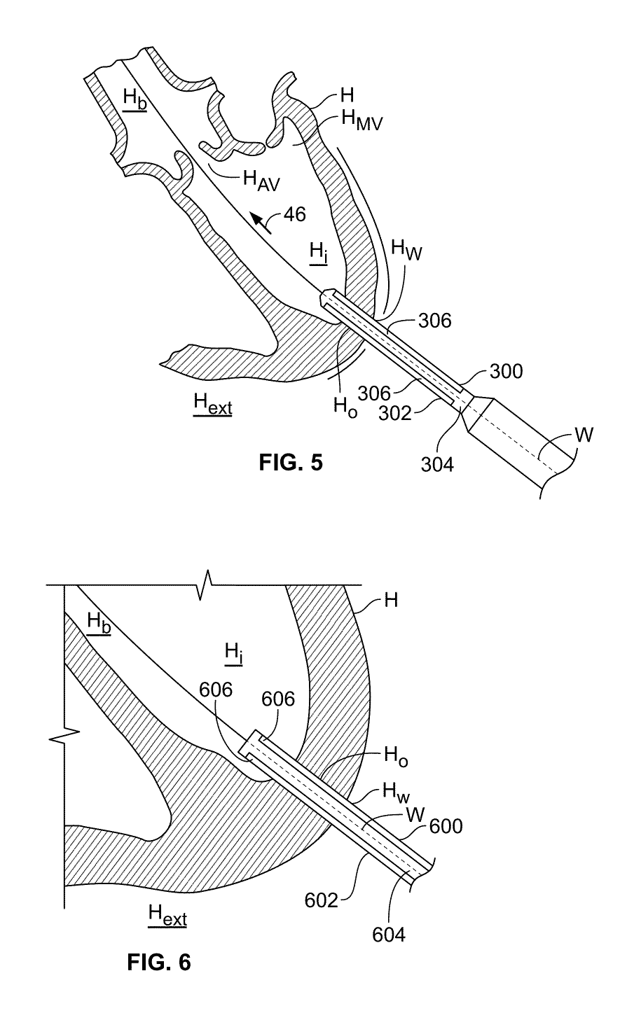

FIG. 5 shows wire W advanced longitudinally through needle 300. Wire W passes through aortic valve H_{AV} into arterial vasculature (not shown). A distal end (not shown) of wire W may be captured by instrumentation (not shown) in the arterial vasculature. Wire W may be advanced through mitral valve Hₘᵥ. Electrically conductive members 306 remain in contact with heart wall H_{w}.

FIG. 6 schematically shows illustrative catheter 600 in access opening Hₒ. Catheter 600 may have replaced needle 300 (shown in FIG. 3). Wire W may have been left in place upon the removal of needle 300. Catheter 600 may have been guided into chamber H_{c} along wire W. Catheter 600 may include elongated member 602. Elongated member 602 may include outer surface 604. Outer surface 604 may resist closure of access opening Hₒ in heart wall H_{w}. The pressure of heart wall H_{w} on outer surface 604 may be sufficient to prevent the leakage of blood H_{b} from interior Hᵢ of chamber H_{c} between outer surface 604 and heart wall H_{w} to exterior Hₑₓₜ of heart H. The pressure may be sufficient to do so even in the absence of a seal at the contact between outer surface 604 and heart wall H_{w}.

Catheter 600 may include one or more electrically conductive members such as representative electrically conductive member 606. One or more of electrically conductive members 606 may be supported by elongated member 602. One or more of electrically conductive members 606 may be supported by outer surface 604. One or more of electrically conductive members 606 may be used to provide electrical pulses to heart wall H_{w} to change the contraction frequency.

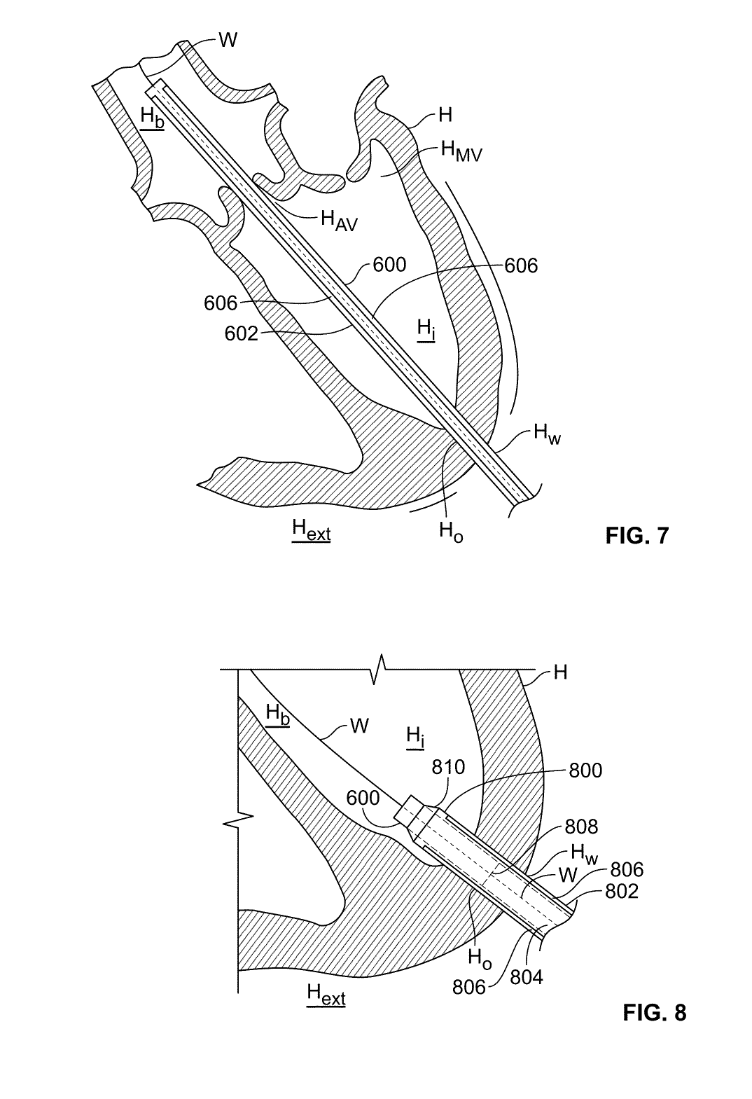

FIG. 7 shows wire catheter 600 (shown in FIG. 6) advanced longitudinally through access opening Hₒ. Catheter 600 passes through aortic valve H_{AV} into arterial vasculature (not shown). Electrically conductive members 606 remain in contact with heart wall H_{w}.

FIG. 8 schematically shows illustrative introducer 800 in access opening Hₒ. Introducer 800 may have been coaxially placed on catheter 600. Catheter 600 may be used to guide introducer 800 into place in access opening Hₒ. Introducer 800 may include elongated member 802. Elongated member 802 may include outer surface 804. Outer surface 804 may resist closure of access opening Hₒ in heart wall H. The pressure of heart wall H_{w} on outer surface 804 may be sufficient to prevent the leakage of blood H_{b} from interior Hᵢ of chamber H_{c} between outer surface 804 and heart wall H_{w} to exterior Hₑₓₜ of heart H. The pressure may be sufficient to do so even in the absence of a seal at the contact between outer surface 804 and heart wall H_{w}.

Introducer 800 may include one or more electrically conductive members such as representative electrically conductive member 806. One or more of electrically conductive members 806 may be supported by elongated member 802. One or more of electrically conductive members 806 may be supported by outer surface 804. One or more of electrically conductive members 806 may be used to provide electrical pulses to heart wall H_{w} to change the contraction frequency. One or more of electrically conductive members 806 may be placed in direct contact with heart wall H_{w} to provide the electrical pulses.

Introducer 800 may advance and withdraw longitudinally through access opening Hₒ. Electrically conductive members 806 remain in contact with heart wall H_{w}.

Introducer 800 may include valve 808. Valve 808 may include an orifice that deforms around an instrument that passes through introducer 800. The instrument may thus be passed through valve 808 while valve 808 reduces or eliminates the loss of blood H_{b} through introducer 800.

Introducer 800 may be used in conjunction with obliterator 810. Obliterator 810 may have one or more features in common with obliterator 101.

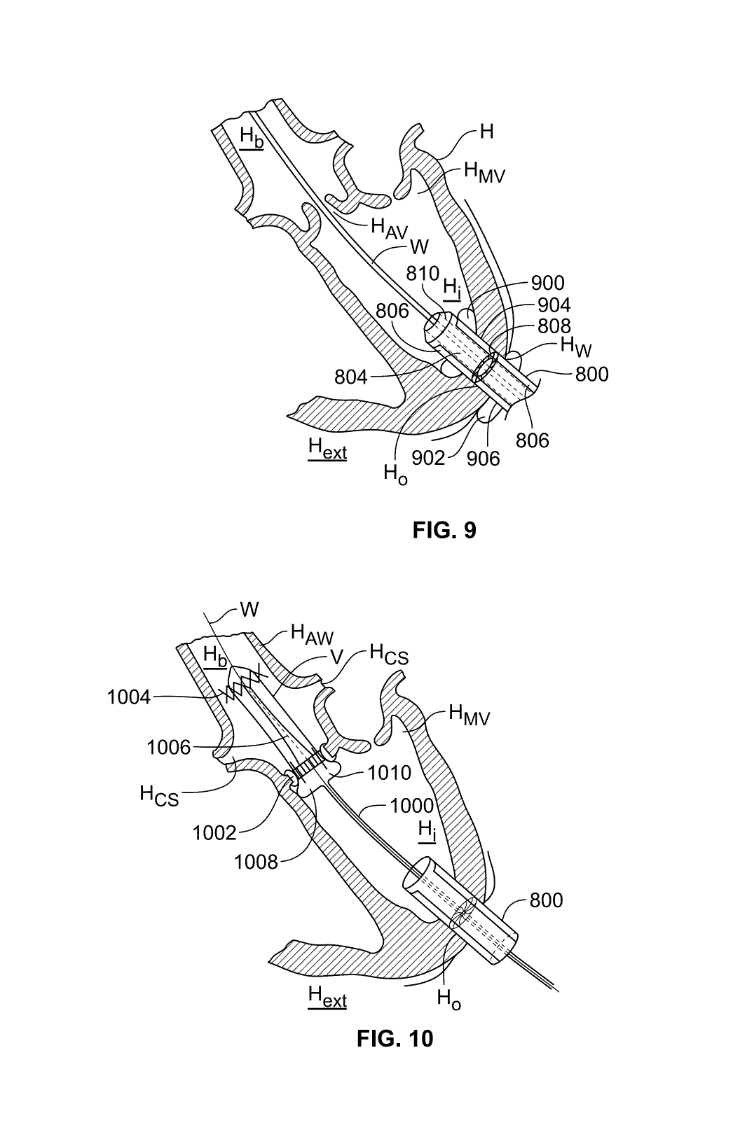

FIG. 9 shows introducer 800 in opening Hₒ after withdrawal of catheter 600. One, both or neither of balloons 900 and 902 may be present adjacent distal and proximal contacts, 904 and 906, respectively, between outer surface 804 and heart wall H. The balloons may interfere with the leakage of blood from heart H.

FIG. 10 shows prosthetic valve V, which has been delivered through introducer 800 and positioned in aortic valve annulus H_{A}. Annulus H_{A} may have resulted from the resection of aortic valve H_{AV} (shown in FIG. 9). The resection may have been performed by deploying resection tools through introducer 800. Valve V may be delivered at the distal tip of a catheter (not shown) such as 600 (shown in FIG. 6).

Valve V may be delivered at the distal tip of the catheter in a procedure that does not involve introducer 800. For example, valve V may fixed to the tip of catheter 600 and advanced distally as catheter 600 is advanced as shown in FIGS. 6 and 7.

FIG. 10 shows valve delivery catheter 1000 extending through introducer 800, although valve delivery catheter 1000 may have one or more features in common with catheter 600 and may be used without an introducer. Catheter 1000 may be advanced and withdrawn through access opening 600 to first deploy proximal fitting 1002 of heart valve V, then stretch distal end 1004 of heart valve V distal of coronary sinuses H_{c}S and secure distal end 1004 to aortic wall H_{AW}. This may properly configure commissure support tissue 1006 to function. Balloon 1008 may be used to radially expand base ring 1010 of valve V. When valve V is deployed without the use of introducer 800, electrically conductive members 606 (shown in FIG. 6) may provide pulses to heart wall H_{w} while catheter 600 is advanced and withdrawn in connection with the deployment of valve V and the withdrawal from heart wall H_{w} of catheter 600.

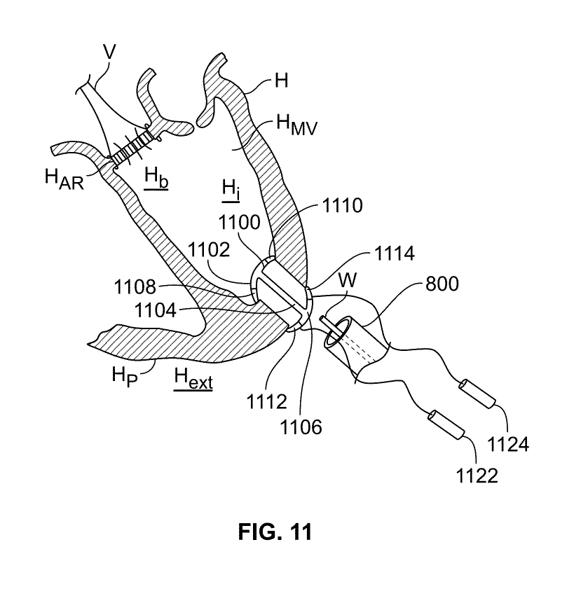

FIG. 11 shows valve V in place in aortic rim H_{AR}. Introducer 800 and wire W have been withdrawn from heart wall H_{w}. Electrically conductive members 806 may have provided pulses to heart wall H_{w} during withdrawal of introducer 800 from heart wall H_{w}. Closure device 1100 may be deployed in access opening Hₒ. Closure device 1100 may provide a seal for access opening Hₒ and may prevent blood H_{b} from exiting access opening Hₒ. The seal may be a temporary seal. The seal may be a permanent seal. Closure device 1100 may be deployed through introducer 800. Closure device 1100 may be deployed without the use of introducer 800.

Closure device 1100 may include distal end 1102. Closure device may include stem 1104. Closure device may include proximal end 1106. Closure device may include one or more electrically conductive members, such as 1108, 1110, 1112 and 1114.

Leads such as 1120, 1122, 1124 and 1126 may provide the electrically conductive members with electrical pulses for conduction to the heart.

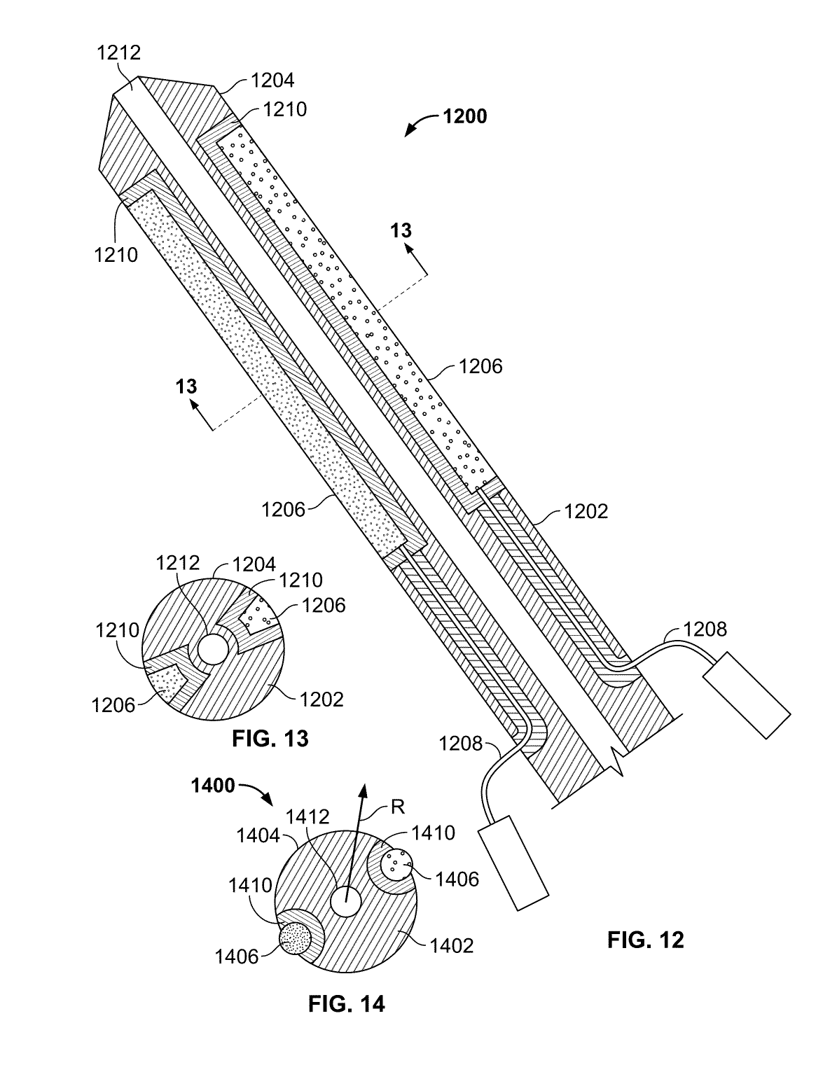

FIG. 12 shows, in a view similar to that which would be seen along lines 12-12 (shown in FIG. 1), an illustrative partial cross-section of instrument 1200. Instrument 1200 may have one or more features in common with instrument 100, needle 300, catheter 600 or introducer 800.

Instrument 1200 may include elongated member 1202. Elongated member 1202 may include outer surface 1204.

Instrument 1200 may include one or more electrically conductive members such as representative electrically conductive member 1206. One or more of electrically conductive members 1206 may be used to provide electrical pulses to heart wall H_{w} to change the contraction frequency. One or more of electrically conductive members 1206 may be placed in direct contact with heart wall H_{w} to provide the electrical pulses. The energy may be supplied by leads such as 1208 from a source such as 110 (shown in FIG. 1). The energy may be supplied wirelessly from the source 110.

Electrically insulating members 1210 may electrically insulate electrically conducting members 1206 from elongated member 1202. When elongated member 1202 is an electrical insulator, electrically insulating members 1210 may not be included.

Instrument 1200 may include delivery lumen 1212 for delivering an instrument to the heart.

FIG. 13 shows, along lines 13-13 (shown in FIG. 12), a cross-section of instrument 1200. Electrically conductive members 1206 and electrically insulating members 1210 may be flush with outer surface 1204 of elongated member 1202.

FIG. 14 shows, in a view similar to that along lines 14-14 (shown in FIG. 12), a cross-section of instrument 1400, which may have one or more features in common with instrument 1200. Instrument 1400 may include elongated member 1402. Elongated member 1402 may include outer surface 1404. Instrument 1400 may include electrically conductive members 1406 and electrically insulating members 1410. Electrically conductive members 1406 may extend radially away from outer surface 1404. Electrically insulating members 1410 may be flush with outer surface 1404. When elongated member 1402 is an electrical insulator, electrically insulating members 1410 may not be included.

Instrument 1400 may include delivery lumen 1412 for delivering an instrument to the heart.

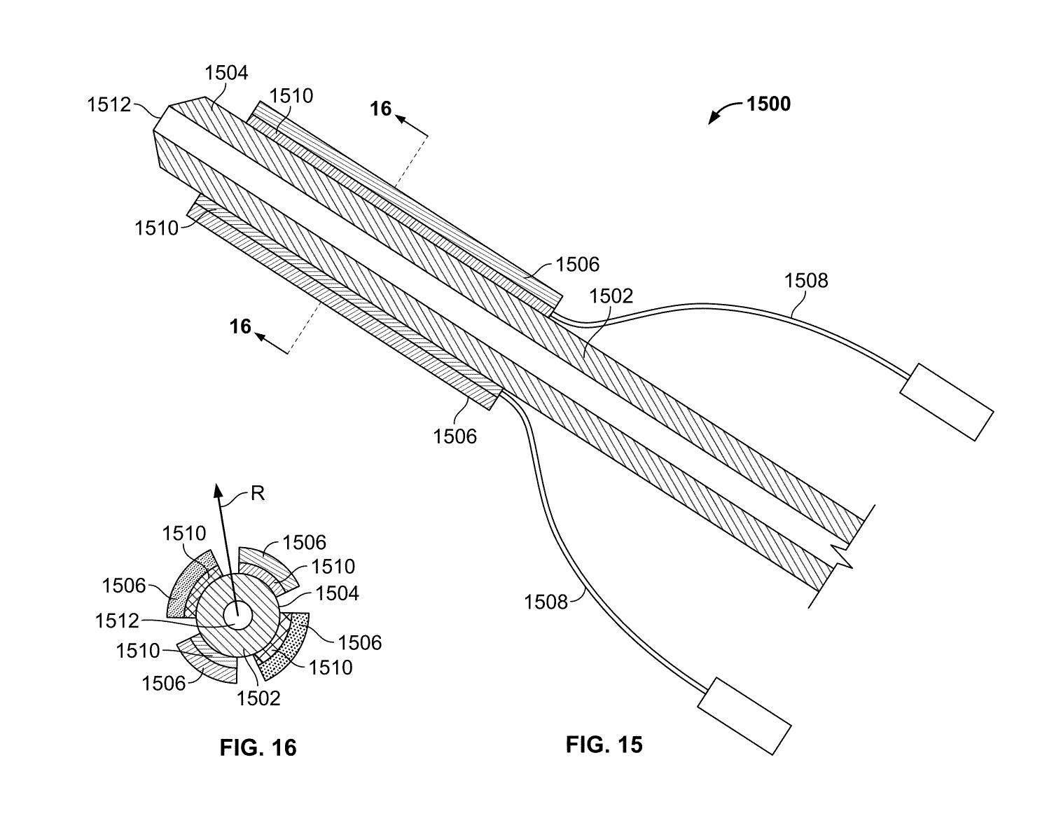

FIG. 15 shows, in a view similar to that which would be seen along lines 15-15 (shown in FIG. 1), an illustrative partial cross-section of instrument 1500. Instrument 1500 may have one or more features in common with instrument 100, needle 300, catheter 600, introducer 800, instrument 1200 or instrument 1300.

Instrument 1500 may include elongated member 1502. Elongated member 1502 may include outer surface 1504.

Instrument 1500 may include one or more electrically conductive members such as representative electrically conductive member 1506. One or more of electrically conductive members 1506 may be used to provide electrical pulses to heart wall H_{w} to change the contraction frequency. One or more of electrically conductive members 1506 may be placed in direct contact with heart wall H_{w} to provide the electrical pulses. The energy may be supplied by leads such as 1508 from a source such as 110 (shown in FIG. 1). The energy may be supplied wirelessly from the source 110.

Electrically insulating members 1510 may electrically insulate electrically conducting members 1506 from elongated member 1502. When elongated member 1502 is an electrical insulator, electrically insulating members 1510 may not be included.

One or more of electrically conducting members 1506 and electrically insulating members 1510 may include tape. One or more of electrically conducting members 1506 and electrically insulating members 1510 may include a thin film. The thin film may be deposited on outer surface 1504. The thin film may deposited on electrically insulating members 1510. The thin film may be printed lithographically.

Instrument 1500 may include delivery lumen 1512 for delivering an instrument to the heart.

FIG. 16 shows, along lines 16-16 (shown in FIG. 15), a cross-section of instrument 1500.

FIG. 17 shows illustrative instrument 1700. Illustrative instrument 1700 may have one or more features in common with instrument 100, needle 300, catheter 600, introducer 800, instrument 1200, instrument 1300 or instrument 1500.

Instrument 1700 may include elongated member 1702. Elongated member 1702 may include outer surface 1704. Outer surface 1704 may resist closure of access opening Hₒ in heart wall H_{w}. The pressure of heart wall H_{w} on outer surface 1704 may be sufficient to prevent the leakage of blood H_{b} from interior Hᵢ of chamber Hₑₓₜ between outer surface 1704 and heart wall H_{w} to exterior Hₑₓₜ of heart H. The pressure may be sufficient to do so even in the absence of a seal at the contact between outer surface 1704 and heart wall H_{w}.

Instrument 1700 may include one or more electrically conductive members such as representative electrically conducting member 1706. One or more of electrically conductive members 1706 may be used to provide electrical pulses to heart wall H_{w} to change the contraction frequency. One or more of electrically conductive members 1706 may be placed in direct contact with heart wall H_{w} to provide the electrical pulses. The energy may be supplied via a cables such as 1708.

Instrument 1700 may include an instrument delivery lumen (not shown). Instrument 1700 may include instrument introduction port 1712. Instrument 1700 may include fluid exchange port 1714.

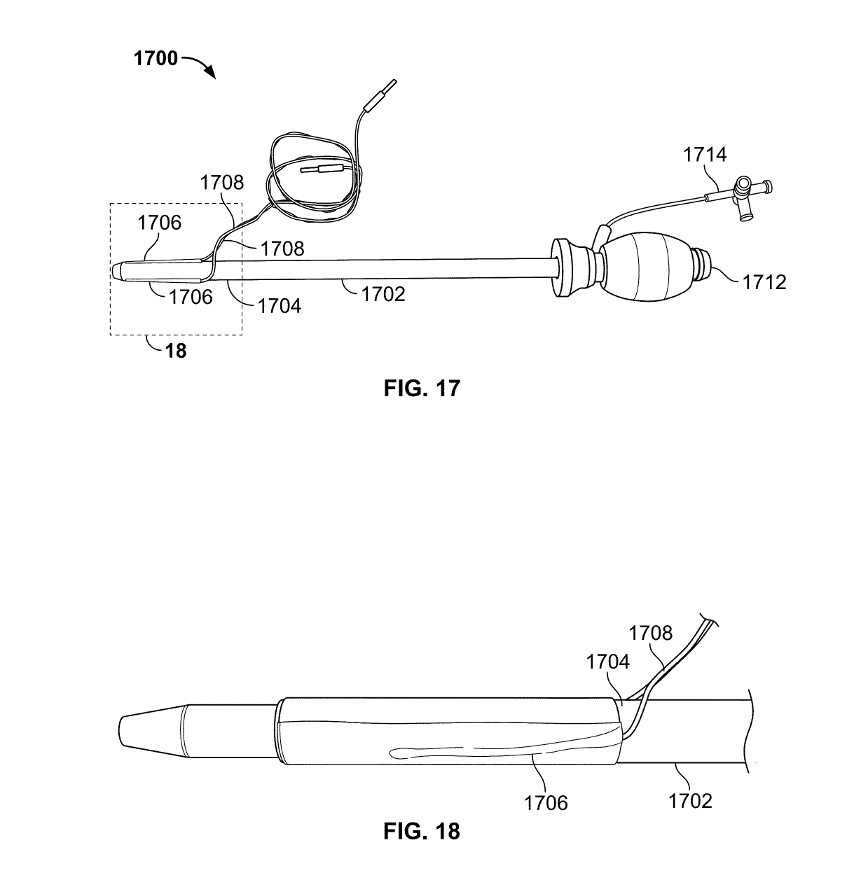

FIG. 18 is a view of region 18 of instrument 1700 (shown in FIG. 17).

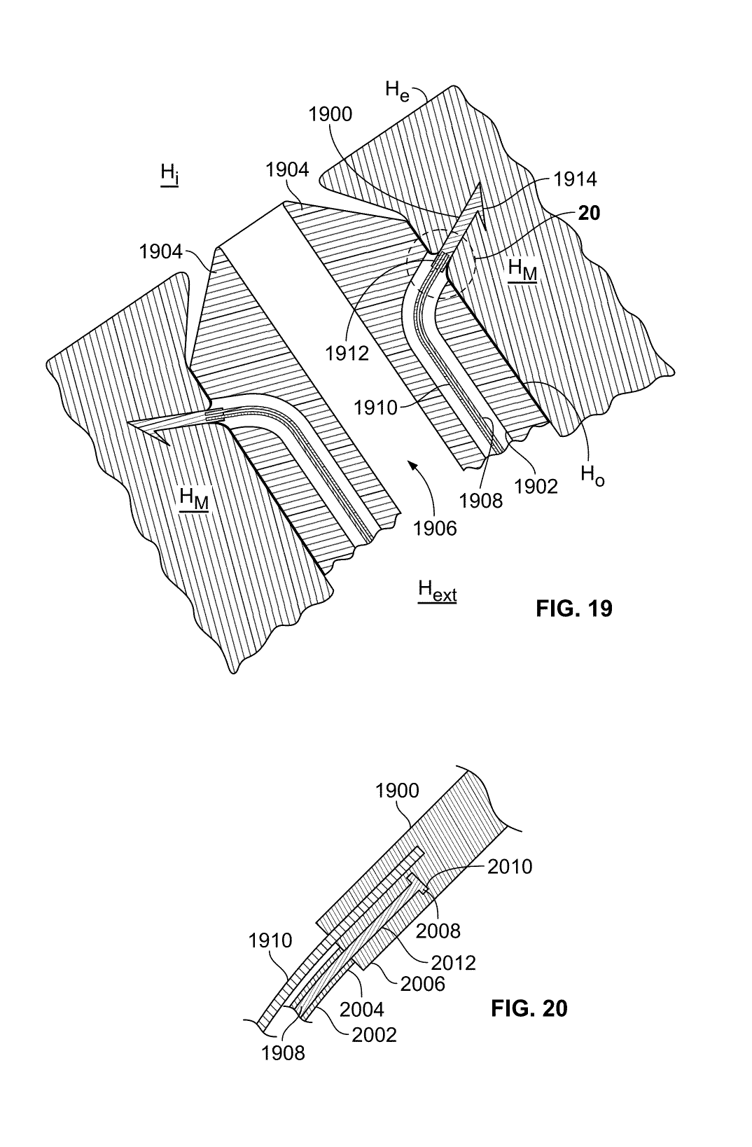

FIG. 19 shows illustrative electrically conductive member 1900 being deployed in myocardium Hₘ of heart H. Electrically conductive member 1900 may be permanently deployed in myocardium Hₘ. Electrically conductive member 1900 may be temporarily deployed in myocardium Hₘ.

Electrically conductive member 1900 may be deployed via deployment lumen 1902 in elongated member 1904. Elongated member 1904 may be part of an instrument such as instrument 100, needle 300, catheter 600, introducer 800, instrument 1200, instrument 1300, instrument 1500, instrument 1700 or instrument 1900.

Elongated member 1904 may positioned by displacing flexible control link 1908 longitudinally along deployment lumen 1902. Flexible control link 1908 may be detachable from electrically conductive member 1900 to deploy electrically conductive member 1900 in heart H. Conductor 1910 may supply the pulses to electrically conductive member 1900. Conductor 1910 may remain attached to electrically conductive member 1900 after control link 1908 is detached from electrically conductive member 1900.

Flexible control link 1908 may be detachably attached to electrically conductive member 1900 via any suitable mechanism such as a key, threaded union, an interference fit, a snap fit, a snap fit with a spring-biased tang or any other suitable mechanism.

Electrically conductive member 1900 may include one or more anchoring mechanisms such as representative flexible barb 1914 or any other suitable anchoring mechanism or substance. For example, the anchoring system may include a spiral thread, circumferential ribs, epoxy, adhesive or any other suitable anchoring mechanism or substance.

FIG. 20 shows illustrative control catheter 2002. Flexible control link 1908 may run through control catheter 2002. Control catheter 2002 may include distal end 2004 that abuts or engages base 2006 of electrically conductive member 1900. Distal end 2004 may provide force to the base so that electrically conductive member 1900 can resist forces associated with the disengagement of flexible control link 1908 from electrically conductive member 1900.

Control link 1908 may engage and disengage electrically conductive member 1900 via “T”-key 2008 in slot 2010. Slot 2012 in electrically conductive member 1900 may be keyed to accommodate passage of “T”-key 2008.

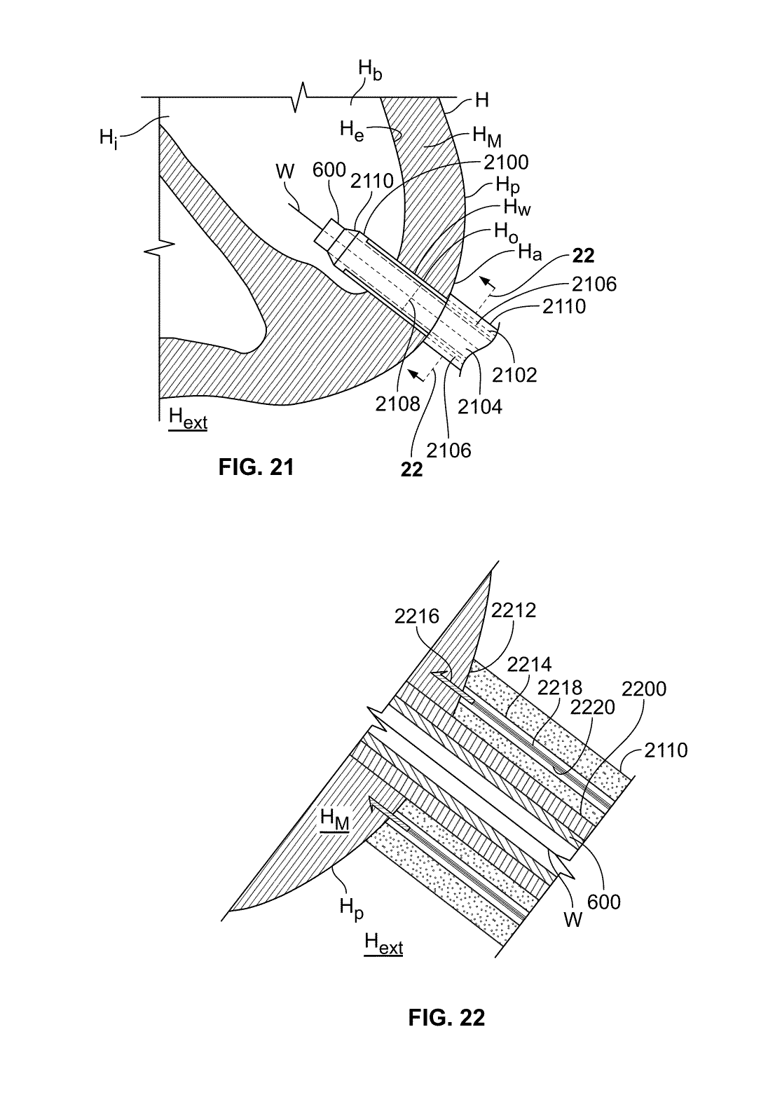

FIG. 21 schematically shows illustrative introducer 2100 in access opening Hₒ. Introducer 2100 may have one or more features in common with instrument 100, needle 300, catheter 600, introducer 800, instrument 1200, instrument 1300, instrument 1500 or instrument 1700.

Introducer 2100 may have been coaxially placed on catheter 600. Catheter 600 may be used to guide introducer 2100 into place in access opening Hₒ. Introducer 2100 may include elongated member 2102. Elongated member 2102 may include outer surface 2104. Outer surface 2104 may resist closure of access opening Hₒ in heart wall H_{w}. The pressure of heart wall H_{w} on outer surface 2104 may be sufficient to prevent the leakage of blood H_{b} from interior Hᵢ of chamber H_{c} between outer surface 2104 and heart wall H_{w} to exterior Hₑₓₜ of heart H. The pressure may be sufficient to do so even in the absence of a seal at the contact between outer surface 2104 and heart wall H_{w}.

Introducer 2100 may include one or more electrically conductive members such as representative electrically conductive member 2106. One or more of electrically conductive members 2106 may be supported by elongated member 2102. One or more of electrically conductive members 2106 may be supported by outer surface 2104. One or more of electrically conductive members 2106 may be used to provide electrical pulses to heart wall H_{w} to change the contraction frequency. One or more of electrically conductive members 2106 may be placed in direct contact with heart wall H_{w} to provide the electrical pulses.

Introducer 2100 may advance and withdraw longitudinally through access opening Hₒ. Electrically conductive members 2106 remain in contact with heart wall H_{w}.

Introducer 2100 may include valve 2108. Valve 2108 may include an orifice that deforms around an instrument that passes through introducer 2100. The instrument may thus be passed through valve 2108 while valve 2108 reduces or eliminates the loss of blood H_{b} through introducer 2100.

Introducer 2100 may be used in conjunction with obliterator 2110. Obliterator 2110 may have one or more features in common with obliterator 101 or obliterator 810.

Illustrative injection catheter 2110 may be provided coaxially about instrument 2100. Injection catheter 2110 may be configured to insert electrically conductive members in heart H. Injection catheter 2110 may be configured to be provided coaxially about one or more of instrument 100, needle 300, catheter 600, introducer 800, instrument 1200, instrument 1300, instrument 1500 and instrument 1700.

FIG. 22 shows in part a partial cross-sectional view of injection catheter 2110 about introducer 2100. Injection catheter 2210 may include distal end 2212. Distal end 2212 may be contoured to conform or partially conform to pericardium Hₚ. Injection catheter may include one or more deployment lumens such as representative deployment lumen 2214. Electrically conducting members such as representative electrically conducting member 2216 may be inserted in myocardium Hₘ using deployment lumens such as representative deployment lumen 2214. Electrically conducting member 2216 may have one or more feature in common with electrically conducting member 2000 (shown in FIG. 20). Injection catheter 2210 may include one or more flexible control link such as representative control link 2218. Flexible control link 2218 may have one or more features in common with flexible control link 2008. Injection catheter 2210 may include one or more conductors such as representative conductor 2220. Conductor 2220 may have one or more features in common with conductor 2010.

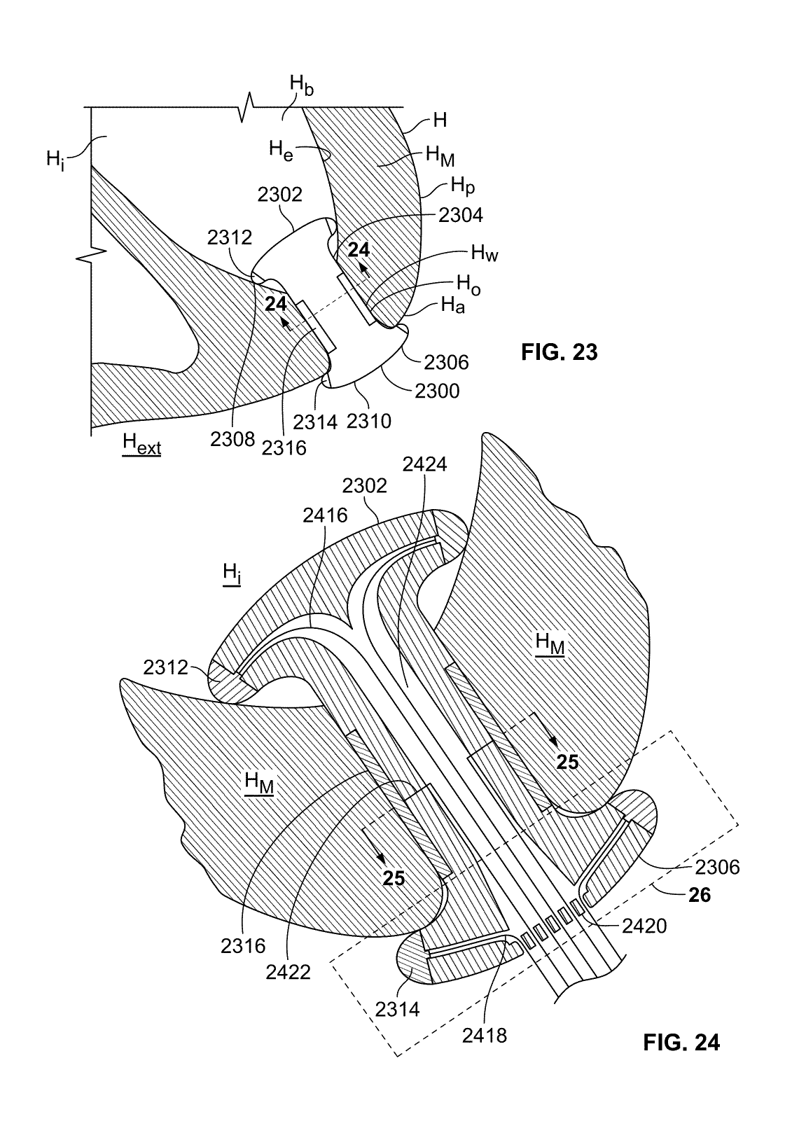

FIG. 23 schematically shows illustrative access opening closure device 2300. Closure device 2300 may have one or more features in common with closure device 1100 (shown in FIG. 11). Closure device 2300 may be inserted in access opening Hₒ. Closure device 2300 may include distal end 2302, stem 2304 and proximal end 2306. One or both of distal end 2302 and proximal end 2306 may have a shape, such as a disc, a cone or a dome to rest flush against endocardium Hₑ or pericardicum Hₚ. The shape reduce or prevent the leakage of blood H_{b} from heart interior Hᵢ. One or more of distal end 2302, stem 2304 and proximal end 2306 may include elastic material. The elastic material may allow stem 2302 to be set in tension across heart wall H_{w}. The tension may compress distal edge 2308 against endocardium Hₑ.

Closure device 2300 may be deployed in access opening Hₒ by delivering distal end 2302 through a cannulated instrument, such as instrument 100 (shown in FIG. 1), when it is cannulated, through access opening Hₒ to heart interior Hᵢ. While retaining proximal end 2306, the distal tip of instrument 100 may be withdrawn from access opening Hₒ and proximal end 2306 may be released in the heart exterior region Hₑₓₜ. Tension in closure device 2300 may then urge proximal end 2306 into position against heart H. Pericardium Hₚ may be retracted so that proximal end 2306 is in contact with myocardium Hₘ.

Closure device 2300 may include body 2310. Body 2310 may include some or all of distal end 2302, stem 2304 and proximal end 2306. Body 2310 may include electrically insulating material such as a polymer that is less electrically conductive than metal or electrically conductive polymer.

Distal end 2302 may include one or more electrically conductive members such as representative conductive member 2312. Electrically conductive member 2312 may extend around the circumference of distal end 2302. Proximal end 2302 may include one or more electrically conductive members such as representative electrically conductive member 2314. Electrically conductive member 2314 may extend around the circumference of distal end 2302.

Stem 2302 may include one or more electrically conductive members such as representative conductive member 2316. The elastic material may allow stem 2302 to be set in radial compression against heart wall H. The compression may maintain electrically conductive member 2316 in contact with heart wall H. Electrically conductive member 2316 may have one or more features in common with electrically conductive member 106 of instrument 100 (shown in FIG. 1).

FIG. 24 shows a cross sectional view of closure device 2300 taken along lines 24-24 (shown in FIG. 23). Electrically conductive members 2312 and 2314 may be in electrical communication with a source such as source 110 (shown in FIG. 1) via conductors 2416 and 2418. Electrically conductive member 2316 may be in electrical communication with a source such as source 110 (shown in FIG. 1) via conductor 2422. The conductors may lead out of closure device 2300 via ported seal 2420. The conductors may run in space 2424, which may be a lumen.

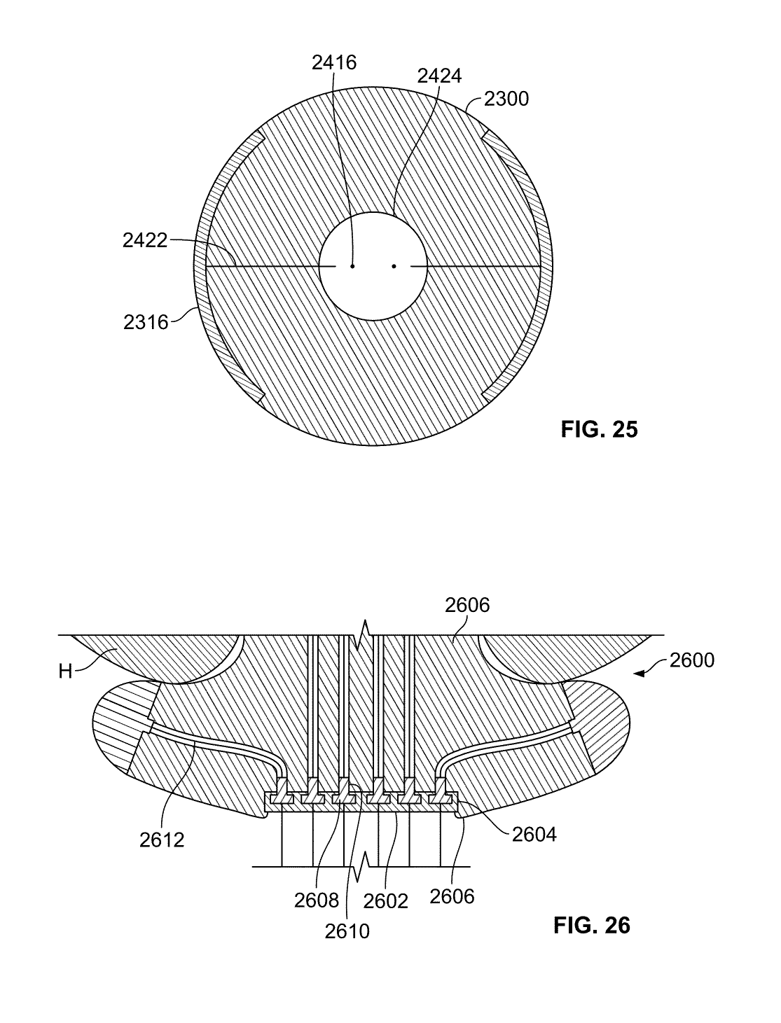

FIG. 25 shows a cross-sectional view of closure device 2300 taken along lines 25-25 (shown in FIG. 24). Electrically conductive member 2316 runs circumferentially around some or all of stem 2304. Conductor 2422 runs radially between space 2424 and electrically conductive member 2316. Conductor 2416 runs longitudinally through space 2424.

FIG. 26 shows in cross section illustrative proximal end 2600 of a closure device such as 2300 (shown in FIG. 23). Proximal end 2600 may have one or more features in common with proximal end 2306 (shown in FIG. 23). Proximal end 2600 may include removable cap 2602. Cap 2602 may be connected to closure device body in recess 2604. Cap 2602 may be secured in recess 2604 by any suitable mechanism such as threads (not shown), a clasp (not shown), a suture (not shown), an interference fit with rib 2606 or any other suitable mechanism.

Cap 2602 may include one or more terminal pins such as representative pin 2608. Terminal pin 2608 may be in electrical communication distally with a source such as 110 (shown in FIG. 1). Terminal pin 2608 may be inserted into receptacle 2610. Terminal pin 2608 may be in electrical communication with receptacle 2610. Receptacle 2610 may be in electrical communication with one or more conductors, such as conductor 2612 that lead to one or more electrically conducting members on body 2606.

The electrically conducting members may have one or more features in common with the electrically conducting members of closure device 2300 (shown in FIG. 23).

Cap 2602 may be removed from distal end 2600. For example, the closure device may be deployed in heart H after delivery of an instrument to interior Hᵢ of heart H. The closure device may remain in heart H while a patient is under postoperative observation. Cap 2602 may be removed. The removal of cap 2602 may disconnect terminal pin 2608 from receptacle 2610. The closure device may thus be disconnected from a source such as 110 (shown in FIG. 1).

A replacement cap (not shown) may be installed in recess 2604. The replacement cap may have one or more non-conducting pins that mate with receptacles such as 4610. The replacement cap may seal against distal end 2600 to prevent fluids from interacting with receptacle 2610. One of the non-conducting pins may seal receptacle 2610. The replacement cap may be permanently installed in recess 2604. The replacement cap may be removably installed in recess 2604. A cap such as 2602 may be installed after removal of the replacement cap to reestablish electrical communication between one or more of the electrically conducting members and a source such as 110 (shown in FIG. 1).

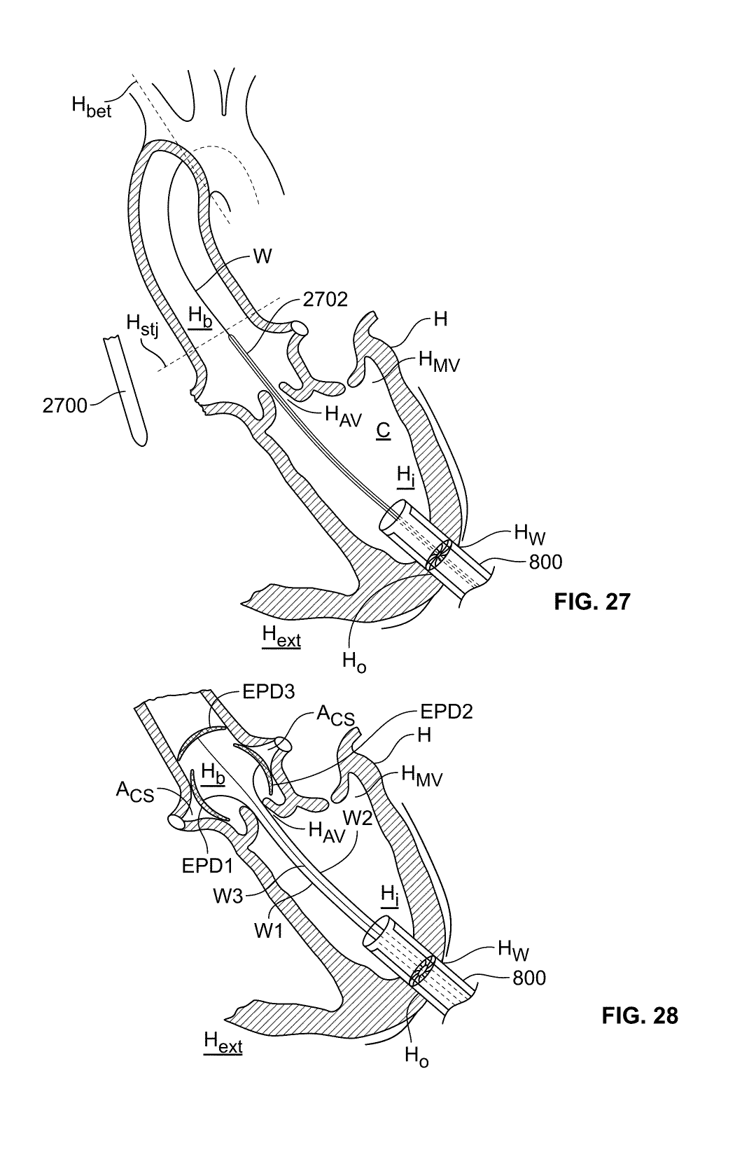

FIG. 27 schematically shows intracardiac ultrasound receiver 2700. Intracardiac ultrasound receiver 2700 may be placed in the right atrium (not shown) via a femoral vein (not shown). Intravascular ultrasound receiver (“IVUS”) 2702 may be delivered into chamber H_{c} over guidewire. IVUS 2702 may be positioned in a valve such as aortic valve H_{AV}.

Any suitable endovascular, endocardiac, and endoluminal visualization aids may be used. Extracorporeal X-ray-based radiographic and fluoroscopic devices may be used map and visualize anatomy and instrumentation related to the procedure.

IVUS 2702 may be used to locate aortic valve H_{AV}, sino-tubular junction H_{STJ}, and brachio-cephalic trunk H_{BCT}. Any suitable analytical mapping platform such as that available under the trademark ACUNAV from Biosense Webster, Inc., of Diamond Bar, Calif., may be used to track IVUS 2702 and determine the location of the anatomical features. The analytical mapping platform may be used in conjunction with fluoroscopy.

A radioopaque marker may be placed on the anatomical features or, in locations corresponding to the anatomical features, on the patient's skin or the heart's surface so that extracorporeal fluoroscopy can later be used to relocate the features.

IVUS 2702 along with the analytical mapping platform and fluoroscopy equipment may be used to take measurements of the diseased valve.

A camera may be inserted through the apparatus into chamber H_{c}. A transparent balloon (filled with a transparent fluid such as water) may be positioned in front of the camera. The camera and liquid-filled balloon are pushed against the surface that the surgeon wishes to view. The transparent balloon displaces blood from the camera's line of sight such that an image of what the camera sees through the balloon is transmitted to the surgeon.

One or more signals from the visualization receivers and devices may be transmitted to a display such as display 120 (shown in FIG. 1).

FIG. 28 shows that an instrument such as 800 (shown in FIG. 8) may deliver to the ascending aorta, near coronary sinuses A_{CS}, illustrative embolic one or more embolic protection devices such as EPD₁, EPD₂ and EPD₃ along guidewires W₁, W₂ and W₃. One or more of EPD₁, EPD₂ and EPD₃ may include a filtering mesh or net made from any suitable material. The chosen material should be able to be collapsed, expanded, and re-collapsed multiple times.

Embolic protection devices may be delivered through introducer 800 for placement in the brachiocephalic, left common carotid, and left subclavian arteries of the aortic arch (not shown).

Introducer 800 may be used to deliver a valve delivery system, such as a catheter on which is mounted an expandable valve, or a temporary valve device (not shown) to a suitable location in the vasculature. For example, when replacing the aortic valve's function, it may be preferable to place the temporary valve in the ascending aorta just distal the native aortic valve. Hₒwever, it is possible to temporarily replace the aortic valve function with a device placed in the descending aorta. Such a placement may have the disadvantage of causing the heart to work harder, but such placements have been proven acceptable in previous surgical procedures.

Introducer 800 may be used to deliver a blood pump such as a ventricular assist device (“VAD”; not shown). The VAD or other temporary pump device may be used to support the heart's natural function while a native valve is being resected or repaired.

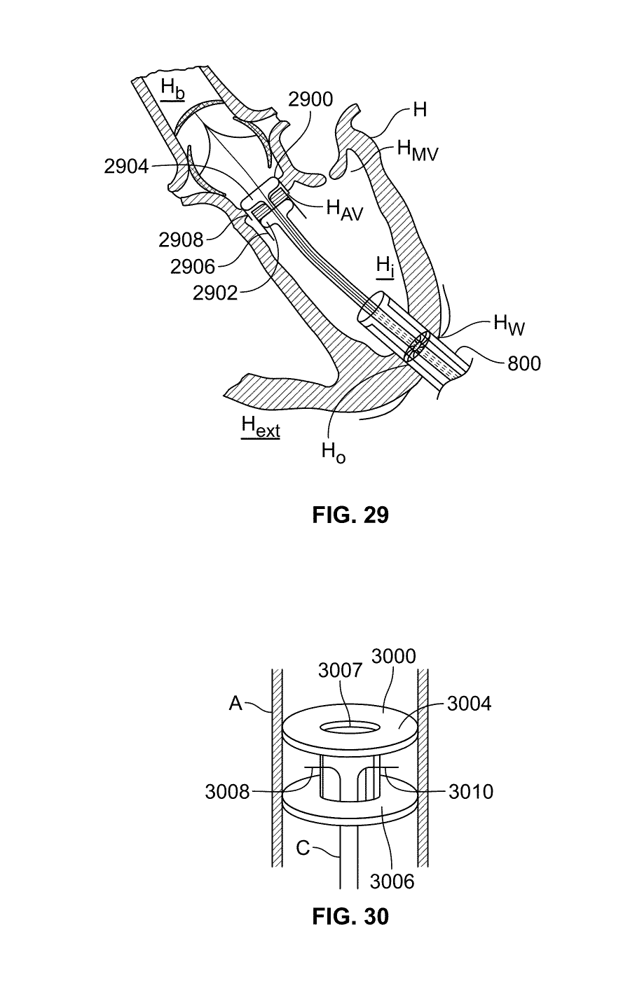

FIG. 29 shows that introducer 800 may be used to deliver balloon-actuated resection tool 2900 to heart H. Tool 2900 may include balloons 2902 and 2904. Balloons 2902 and 2904 may drive together radially expanded cutting edges 2906 and 2908 to resect a valve such as aortic valve H_{AV}.

FIG. 30 shows illustrative ablation chamber 3000 that may be delivered to heart H using introducer 800. Ablation chamber 3000 is shown deployed in aorta A. Ablation chamber 3000 may include distal containment barrier 3004 and proximal containment barrier 3006. Cather C may have one or more lumens. One or more of the lumens may permit blood circulation through lumen 3007. One or more of the lumens may supply outlet 3008 with an ablation chemical. One or more of the lumens may receive ablation material from inlet 3010. A laser ablation device (not shown) may be delivered to heart H using introducer 800.

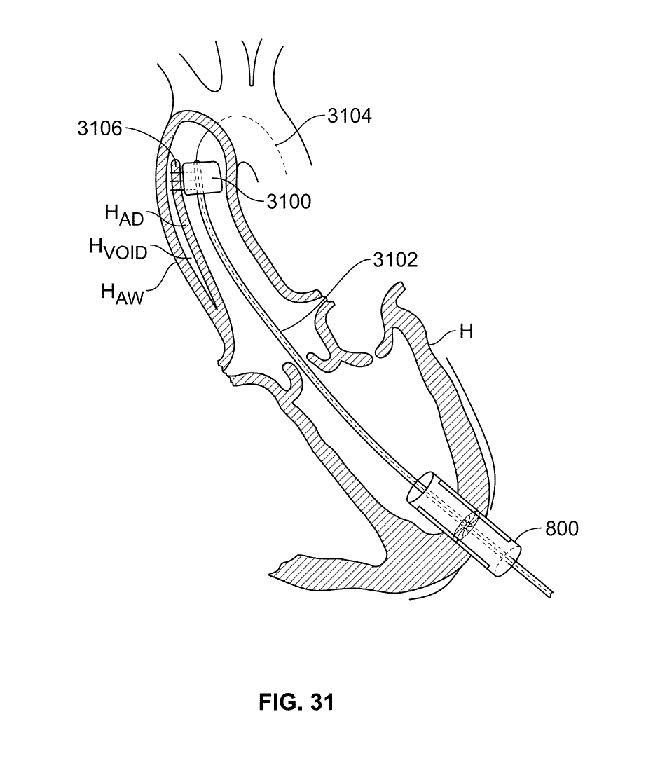

FIG. 31 shows illustrative fluid delivery balloon assembly 3100 that may be delivered to heart H using introducer 800. Assembly 3100 may be supported by one or both of catheter 3102 and wire 3104. Assembly 3100 may include one or more cannulated needles such as representative cannulated needle 3106. Cannulated needle 3106 may deliver a fluid to heart H. For example, the fluid may include glue. Needle 3106 may penetrate aortic dissection H_{AD} such that a tips of needle 3106 is exposed in void H_{void}.

The glue may be a biologically compatible glue. The glue may be injected through needle 3106 via a glue delivery lumen (not shown) in catheter 3102. Inflation of a balloon in assembly 3100 may ensure that dissection H_{AD} is securely affixed to aorta wall H_{AW} by the biologically compatible glue.

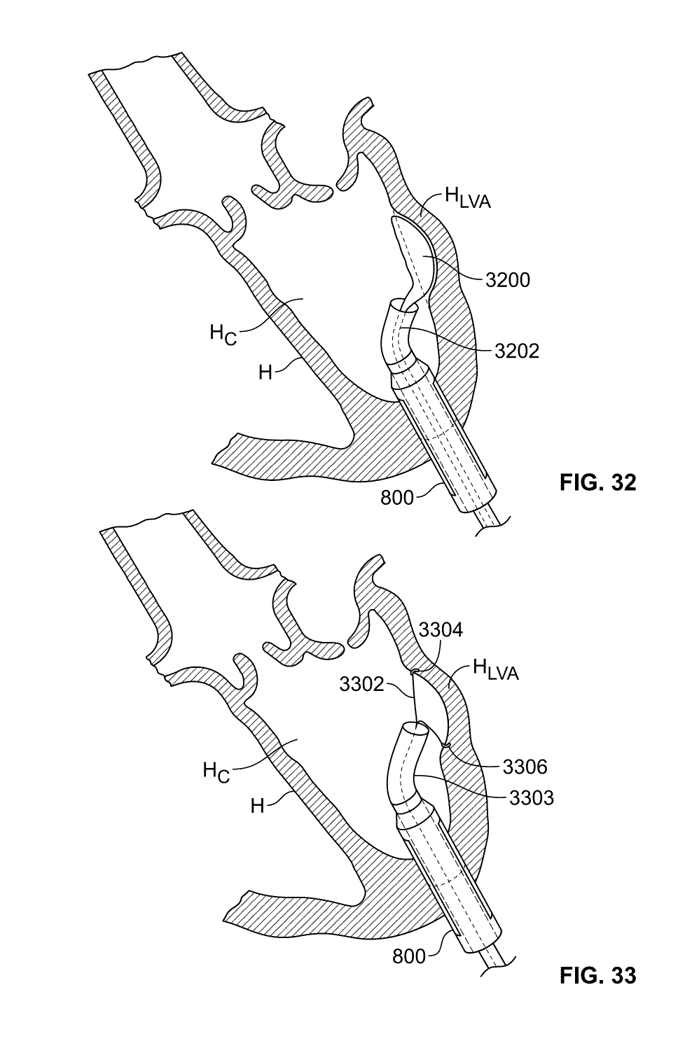

FIG. 32 shows illustrative fluid filled bolster 3200 that may be delivered to heart H using introducer 800. Bolster 3200 may be deployed through catheter 3202. Bolster 3200 may be used to repair, stabilize or fill in left ventricular aneurysm H_{LVA} or any other aneurysm. Bolster 3200 may be installed in aneurysm H_{LVA} using the glue, sutures, clips or by any other suitable mounting technique.

FIG. 33 shows illustrative repair device 3302 that may be delivered to heart H using introducer 800. Device 3302 may be deployed through catheter 3303. Device 3302 may include one or more hooks such as 3304 and 3306. Aneurysm H_{LVA} may be repaired by using the hooks to pull ends of aneurysm H_{LVA} together. Hooks 3304 and 3306 may grasp the interior of heart H at extremes of aneurysm H_{LVA} and then draw aneurysm H_{LVA} closed. Any suitable technique can be used to secure aneurysm H_{LVA} in the closed position (e.g., biologically compatible glue, surgical staples, mechanically placed sutures, etc.). Once the aneurysm has been sealed, repair device 3302 may be withdrawn from the patient.

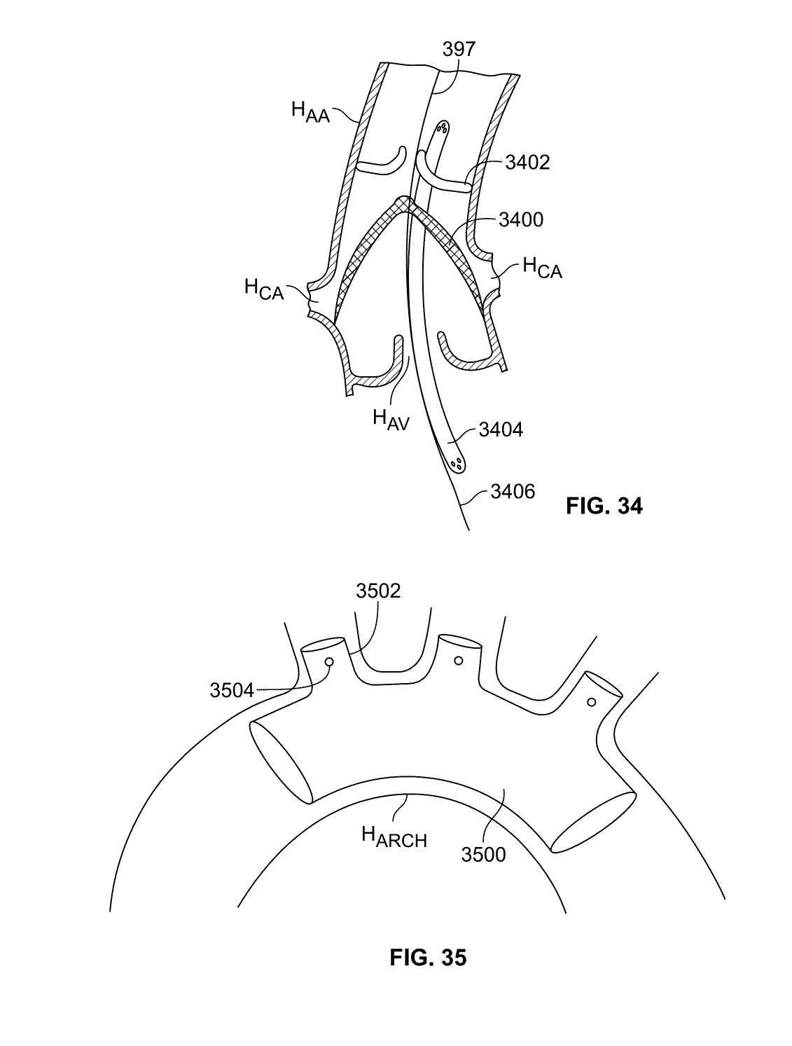

FIG. 34 shows illustrative embolic filter 3400, temporary valve 3402 and VAD 3404 that may be delivered to heart H using introducer 800 (shown in FIG. 8). One or more of embolic filter 3400, temporary valve 3402 and VAD 3404 may be positioned using wire 3406. Wire 3406 may be delivered to heart chamber H_{c} using introducer 800. One or more of embolic filter 3400, temporary valve 3402 and VAD 3404 may be positioned distal aortic valve H_{AV} in in ascending aorta H_{AA}. Embolic filter 3400 may be configured to protect coronary arteries H_{CA}.

FIG. 35 shows illustrative endoprosthesis 3500 that may be delivered to heart H using introducer 800 (shown in FIG. 8). Endoprosthesis 3500 is illustrated as being configured for deployment in aortic arch H_{arch}, but endoprothesis 3500 may be configured to treat many different anatomical structure. Endoprosthesis 3500 may include arms such as 3502 that extend into the brachiocephalic artery, left common carotid artery, and left subclavian artery 408. Endoprosthesis 3500 may be placed using a guidewire that may pass through introducer 800 and engage a hole such as 3504. In order to aid the insertion of the arms into the arterial branches, small catheters, or other pushing devices, may be delivered through introducer 800 and inserted over guidewires to push the arms. Endoprosthesis 3500 and arm 3502 may be radially expanded once endoprosthesis 3500 is properly positioned.

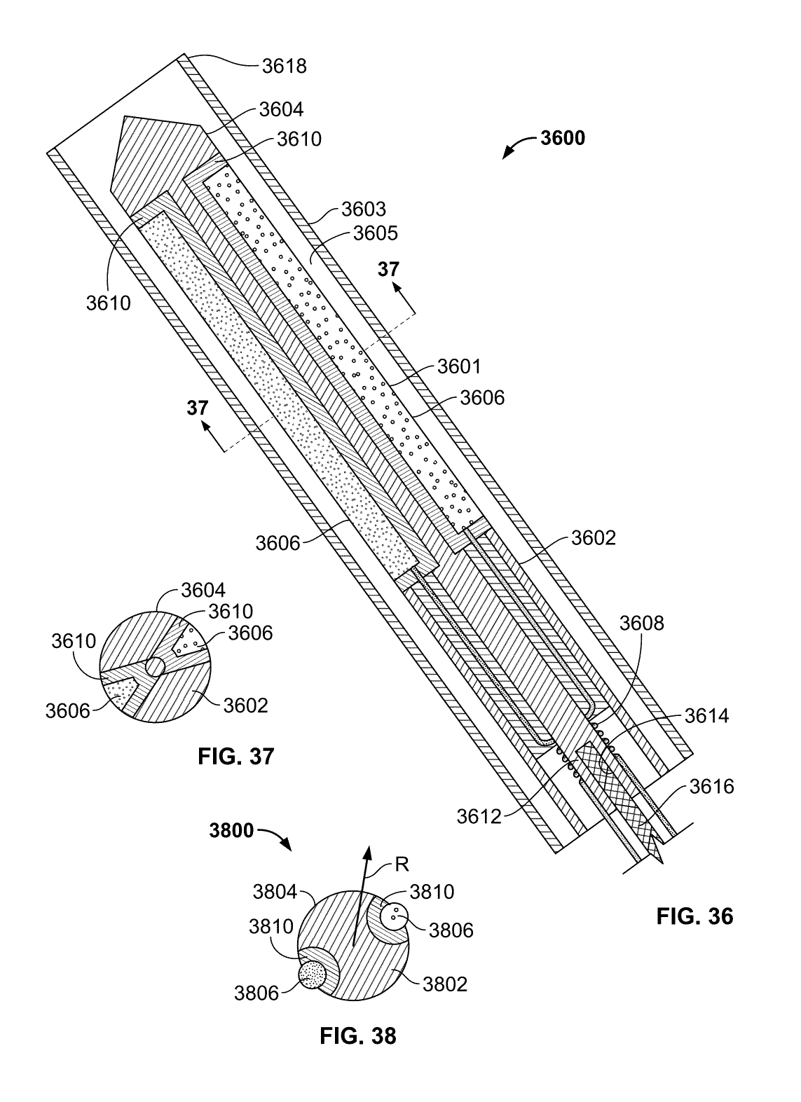

FIG. 36 shows a cross section of illustrative needle assembly 3600. Needle assembly 3600 may include needle 3601. Illustrative needle 3601 may have one or more features in common with instrument 100, needle 300, catheter 600, introducer 800, instrument 1200, instrument 1300, instrument 1500 or instrument 1700.

Needle assembly 3600 may include firing tube 3603. Firing tube 3603 may include lumen 3605. A firing apparatus (which may include firing tube 3603 and peg 3616, but otherwise is not shown) may act on firing post 3616 to eject needle 3601 from tube 3603 and into myocardium Hₘ (not shown). The insertion of needle 3601 may be the establishment of an access opening such as Hₒ (shown in FIG. 1).

Distal end 3618 of firing tube 3603 may be placed adjacent or within an intercostal region. Firing tube may be angled toward heart Hₘ. Distal end 3618 of firing tube 3603 may be placed on heart Hₘ. Distal end 3618 of firing tube 3603 may be placed on the skin.

The firing apparatus may be used to insert needle 3601 into myocardium Hₘ is a surgical setting in which a surface of heart (H) is exposed, in a percutaneous setting in which access to heart H is provided by a small incision or in a strictly percutaneous setting in which needle 3601 is ejected from tube 3603 through the skin and into myocardium Hₘ.

Needle 3601 may include elongated member 3602. Elongated member 3602 may be cannulated. Elongated member 3602 may be uncannulated. Elongated member may not include a delivery lumen.

Elongated member 3602 may include outer surface 3604. Outer surface 3604 may resist closure of access opening Hₒ (shown in FIG. 1) in heart wall H_{w} (shown in FIG. 1). The pressure of heart wall H_{w} on outer surface 3604 may be sufficient to prevent the leakage of blood H_{b} from interior Hᵢ of chamber H_{c} between outer surface 3604 and heart wall H_{w} to exterior Hₑₓₜ of heart H. The pressure may be sufficient to do so even in the absence of a seal at the contact between outer surface 3604 and heart wall H_{w}.

Needle 3601 may include one or more electrically conductive members such as 3606. One or more of electrically conductive members 3606 may be supported by elongated member 3602. One or more of electrically conductive members 3606 may be used to provide electrical pulses to heart wall H_{w} to change the contraction frequency. One or more of electrically conductive members 3606 may be placed in direct contact with heart wall H_{w} to provide the electrical pulses. The energy may be supplied via a cable such as 3608 from a source such as 110 (shown in FIG. 1). The energy may be supplied wirelessly from source 110. An electrically conductive member may be placed on the skin or on the heart surface to complement current supplied by electrically conductive member 3606.

Electrically insulating members 3610 may electrically insulate electrically conducting members 3606 from elongated member 3602. When elongated member 3602 is an electrical insulator, electrically insulating members 3610 may not be included.

Cable 3608 may be wound about firing post 3612. A driving force may be delivered to receptacle 3614 inside post 3612. The driving force may be supplied by peg 3616. Peg 3616 may be driven by any suitable mechanism. For example, peg 3616 may be driven by a spring or compressed gas or by hand. Peg 3616 may drive needle 3612 longitudinally and allow needle 3612 to disengage from peg 3616 and penetrate myocardium Hₘ. Needle 3601 may be driven directly by the spring or compressed gas or by the hand.

When needle 3601 is driven distally, cable 3608 may unravel from drive post 3612. A practitioner may pull on cable 3608 to evaluate whether needle 3612 has become lodged in myocardium Hₘ.

Needle assembly 3600 may include more than one firing tube. Each firing tube may include one needle. When each needle includes one electrically conductive member, the two or more needles may provide current to different locations in the heart. The different locations may be proximate each other, for example, within 1-5 needle diameters of each other. The different locations may be apart from each other, for example, within 6-10, 10-20 or more needle diameters from each other.

One or more additional electrically conductive members may have a distal end that is placed in electrical communication with epidermal tissue on the body in which the heart is disposed. The one or more additional electrically conductive members may have a proximal end that is in electrical communication with control panel 3612.

FIG. 37 shows, along lines 37-37 (shown in FIG. 36), a cross-section of needle 3601. Electrically conductive members 3606 and electrically insulating members 3610 may be flush with outer surface 3604 of elongated member 3602.

FIG. 38 shows, in a view similar to that along lines 36-36 (shown in FIG. 36), a cross-section of instrument 3800, which may have one or more features in common with needle 3601. Instrument 3800 may include elongated member 3802. Elongated member 3802 may include outer surface 3804. Instrument 3800 may include electrically conductive members 3806 and electrically insulating members 3810. Electrically conductive members 3806 may extend radially away from outer surface 3804. Electrically insulating members 3810 may be flush with outer surface 3804. When elongated member 3802 is an electrical insulator, electrically insulating members 3810 may not be included.

Thus, apparatus and methods for delivering an instrument through an access opening in an exterior heart wall have been provided. Persons skilled in the art will appreciate that the present invention can be practiced by other than the described embodiments, which are presented for purposes of illustration rather than of limitation.

The present invention is limited only by the claims that follow.

What is claimed is:

1. Apparatus for delivering an instrument through an access opening in an exterior heart wall, the heart wall having a contraction frequency, the apparatus comprising:
an elongated member having:
a delivery lumen that is configured to traverse the heart wall, the lumen having a proximal opening for receiving the instrument and a distal opening for deploying the instrument; and
an outer surface that is configured to maintain the access opening; and
an electrically conductive member that is supported by the elongated member and is configured to deliver to the heart wall a current that modifies the frequency.

2. The apparatus of claim 1 wherein the electrically conductive member is configured to provide to the heart wall a series of pulses.

3. The apparatus of claim 1 wherein the electrically conductive member is configured to be in direct contact with the heart wall.

4. The apparatus of claim 1 wherein the electrically conductive member is configured to slide through the access opening while delivering the current.

5. The apparatus of claim 1 further comprising an antenna that is supported by the elongated member and is configured to sense a native cardiac electric field in a chamber interior the heart wall and communicate a corresponding native cardiac signal to a receiver exterior the heart wall.

6. The apparatus of claim 1 further comprising a pressure sensor that is configured to sense a pressure in a chamber interior the heart wall and transmit a corresponding pressure signal to a receiver exterior the heart wall.

7. The apparatus of claim 1 further comprising a chemical sensor that is configured to sense a chemical species at a myocardial tissue surface that is exposed in a heart wall access opening and transmit a corresponding chemical signal to a receiver exterior the heart wall.

8. The apparatus of claim 17 wherein the electrically conductive member is configured to be released from the elongated member and inserted in the heart wall.

9. The apparatus of claim 1 wherein:
when a portion of the elongated member is in a contracted state, a portion of the electrically conductive member is disposed at a first radial distance from a longitudinal central axis of the delivery lumen; and
when the portion of the elongated member is in an expanded state, the portion of the electrically conductive member is disposed at a second radial distance from the axis, the second radial distance being greater than the first radial distance.

10. The apparatus of claim 9 wherein:
the portion of the electrically conductive member is a first portion; and
a second portion of the electrically conductive member, disposed longitudinally away from the first portion, is disposed at the first radial distance from the axis.

11. Apparatus for plugging an access opening in a heart wall of a heart, the apparatus comprising:
a body that includes:
a distal end that is configured to be disposed interior the heart and contact endocardial tissue adjacent the access opening; and
a proximal end that is configured to be disposed exterior the heart and contact heart tissue adjacent the access opening; and
an electrode that is supported by the body and is configured to discharge electrical energy into the heart wall to change a frequency of contraction of the heart.

12. The apparatus of claim 11 further comprising a stem that extends between the distal end and the proximal end.

13. The apparatus of claim 11 wherein:
the stem has a first diameter;
the distal end has a second diameter that is greater than the first diameter; and
the proximal end has a third diameter that is greater than the first diameter.

14. The apparatus of claim 11 wherein the electrode discharges from the distal end.

15. The apparatus of claim 11 wherein the electrode discharges from the proximal end.

16. The apparatus of claim 11 further comprising, when the electrode is a first electrode, a second electrode that is configured to discharge electrical energy into the heart wall to change the frequency of contraction of the heart.

17. The apparatus of claim 11 further comprising a sensor for sensing a native cardiac electric field.

18. A method for delivering an instrument to the interior of a heart, the method comprising:
inserting an elongated member through an access opening in the heart;
transferring electrical energy from the elongated member to the heart to change a frequency of contractions of the heart; and
delivering an instrument through the elongated member.

19. Apparatus for pacing a heart, the apparatus comprising:
a sharp end that is configured to pierce tissue;
a base opposite the sharp end, the base being configured to receive an impulse to drive the sharp end into the tissue;
an elongated member bearing an electrode, the elongated member extending from the sharp end to the base; and
an electrically conducting lead that is connected to the electrode and is configured to receive current for delivery to the heart through the electrode when the electrode is embedded in the heart.

20. The apparatus of claim 19 further comprising, when the electrode is a first electrode and the electrically conducting lead is a first electrically conducting lead:
a second electrode that is borne by the elongated member; and
a second electrically conducting lead that is connected to the electrode and is configured to receive current for delivery to the heart through the second electrode when the second electrode is embedded in the heart.
